SEQUENCE LISTING (7 sequences)

SEQ ID NO: 1 - protein, 2003 aa, Homo sapiens
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 2 - DNA, 6012 bp, Homo sapiens
atgcagcccccttcactgctgctgctgctgctgctgctgctgctgctatgtgtctcagtggtcagacccagagggctgctgtgtgggagtttcccagaaccctgtgccaatggaggcacctgcctgagcctgtctctgggacaagggacctgccagtgtgcccctggcttcctgggtgagacgtgccagtttcctgacccctgccagaacgcccagctctgccaaaatggaggcagctgccaagccctgcttcccgctcccctagggctccccagctctccctctccattgacacccagcttcttgtgcacttgcctccctggcttcactggtgagagatgccaggccaagcttgaagacccttgtcctccctccttctgttccaaaaggggccgctgccacatccaggcctcgggccgcccacagtgctcctgcatgcctggatggacaggtgagcagtgccagcttcgggacttctgttcagccaacccatgtgttaatggaggggtgtgtctggccacatacccccagatccagtgccactgcccaccgggcttcgagggccatgcctgtgaacgtgatgtcaacgagtgcttccaggacccaggaccctgccccaaaggcacctcctgccataacaccctgggctccttccagtgcctctgccctgtggggcaggagggtccacgttgtgagctgcgggcaggaccctgccctcctaggggctgttcgaatgggggcacctgccagctgatgccagagaaagactccacctttcacctctgcctctgtcccccaggtttcataggcccagactgtgaggtgaatccagacaactgtgtcagccaccagtgtcagaatgggggcacttgccaggatgggctggacacctacacctgcctctgcccagaaacctggacaggctgggactgctccgaagatgtggatgagtgtgagacccagggtccccctcactgcagaaacgggggcacctgccagaactctgctggtagctttcactgcgtgtgtgtgagtggctggggcggcacaagctgtgaggagaacctggatgactgtattgctgccacctgtgccccgggatccacctgcattgaccgggtgggctctttctcctgcctctgcccacctggacgcacaggactcctgtgccacttggaagacatgtgtctgagccagccgtgccatggggatgcccaatgcagcaccaaccccctcacaggctccacactctgcctgtgtcagcctggctattcggggcccacctgccaccaggacctggacgagtgtctgatggcccagcaaggcccaagtccctgtgaacatggcggttcctgcctcaacactcctggctccttcaactgcctctgtccacctggctacacaggctcccgttgtgaggctgatcacaatgagtgcctctcccagccctgccacccaggaagcacctgtctggacctacttgccaccttccactgcctctgcccgccaggcttagaagggcagctctgtgaggtggagaccaacgagtgtgcctcagctccctgcctgaaccacgcggattgccatgacctgctcaacggcttccagtgcatctgcctgcctggattctccggcacccgatgtgaggaggatatcgatgagtgcagaagctctccctgtgccaatggtgggcagtgccaggaccagcctggagccttccactgcaagtgtctcccaggctttgaagggccacgctgtcaaacagaggtggatgagtgcctgagtgacccatgtcccgttggagccagctgccttgatcttccaggagccttcttttgcctctgcccctctggtttcacaggccagctctgtgaggttcccctgtgtgctcccaacctgtgccagcccaagcagatatgtaaggaccagaaagacaaggccaactgcctctgtcctgatggaagccctggctgtgccccacctgaggacaactgcacctgccaccacgggcactgccagagatcctcatgtgtgtgtgacgtgggttggacggggccagagtgtgaggcagagctagggggctgcatctctgcaccctgtgcccatggggggacctgctacccccagccctctggctacaactgcacctgccctacaggctacacaggacccacctgtagtgaggagatgacagcttgtcactcagggccatgtctcaatggcggctcctgcaaccctagccctggaggctactactgcacctgccctccaagccacacagggccccagtgccaaaccagcactgactactgtgtgtctgccccgtgcttcaatgggggtacctgtgtgaacaggcctggcaccttctcctgcctctgtgccatgggcttccagggcccgcgctgtgagggaaagctccgccccagctgtgcagacagcccctgtaggaatagggcaacctgccaggacagccctcagggtccccgctgcctctgccccactggctacaccggaggcagctgccagactctgatggacttatgtgcccagaagccctgcccacgcaattcccactgcctccagactgggccctccttccactgcttgtgcctccagggatggaccgggcctctctgcaaccttccactgtcctcctgccagaaggctgcactgagccaaggcatagacgtctcttccctttgccacaatggaggcctctgtgtcgacagcggcccctcctatttctgccactgcccccctggattccaaggcagcctgtgccaggatcacgtgaacccatgtgagtccaggccttgccagaacggggccacctgcatggcccagcccagtgggtatctctgccagtgtgccccaggctacgatggacagaactgctcaaaggaactcgatgcttgtcagtcccaaccctgtcacaaccatggaacctgtactcccaaacctggaggattccactgtgcctgccctccaggctttgtggggctacgctgtgagggagacgtggacgagtgtctggaccagccctgccaccccacaggcactgcagcctgccactctctggccaatgccttctactgccagtgtctgcctggacacacaggccagtggtgtgaggtggagatagacccctgccacagccaaccctgctttcatggagggacctgtgaggccacagcaggatcacccctgggtttcatctgccactgccccaagggttttgaaggccccacctgcagccacagggccccttcctgcggcttccatcactgccaccacggaggcctgtgtctgccctcccctaagccaggcttcccaccacgctgtgcctgcctcagtggctatgggggtcctgactgcctgaccccaccagctcctaaaggctgtggccctccctccccatgcctatacaatggcagctgctcagagaccacgggcttggggggcccaggctttcgatgctcctgccctcacagctctccagggccccggtgtcagaaacccggagccaaggggtgtgagggcagaagtggagatggggcctgcgatgctggctgcagtggcccgggaggaaactgggatggaggggactgctctctgggagtcccagacccctggaagggctgcccctcccactctcggtgctggcttctcttccgggacgggcagtgccacccacagtgtgactctgaagagtgtctgtttgatggctacgactgtgagacccctccagcctgcactccagcctatgaccagtactgccatgatcacttccacaacgggcactgtgagaaaggctgcaacactgcagagtgtggctgggatggaggtgactgcaggcctgaagatggggacccagagtgggggccctccctggccctgctggtggtactgagccccccagccctagaccagcagctgtttgccctggcccgggtgctgtccctgactctgagggtaggactctgggtaaggaaggatcgtgatggcagggacatggtgtacccctatcctggggcccgggctgaagaaaagctaggaggaactcgggaccccacctatcaggagagagcagcccctcaaacgcagcccctgggcaaggagaccgactccctcagtgctgggtttgtggtggtcatgggtgtggatttgtcccgctgtggccctgaccacccggcatcccgctgtccctgggaccctgggcttctactccgcttccttgctgcgatggctgcagtgggagccctggagcccctgctgcctggaccactgctggctgtccaccctcatgcagggaccgcaccccctgccaaccagcttccctggcctgtgctgtgctccccagtggccggggtgattctcctggccctaggggctcttctcgtcctccagctcatccggcgtcgacgccgagagcatggagctctctggctgccccctggtttcactcgacggcctcggactcagtcagctccccaccgacgccggcccccactaggcgaggacagcattggtctcaaggcactgaagccaaaggcagaagttgatgaggatggagttgtgatgtgctcaggccctgaggagggagaggaggtgggccaggctgaagaaacaggcccaccctccacgtgccagctctggtctctgagtggtggctgtggggcgctccctcaggcagccatgctaactcctccccaggaatctgagatggaagcccctgacctggacacccgtggacctgatggggtgacacccctgatgtcagcagtttgctgtggggaagtacagtccgggaccttccaaggggcatggttgggatgtcctgagccctgggaacctctgctggatggaggggcctgtccccaggctcacaccgtgggcactggggagacccccctgcacctggctgcccgattctcccggccaaccgctgcccgccgcctccttgaggctggagccaaccccaaccagccagaccgggcagggcgcacaccccttcatgctgctgtggctgctgatgctcgggaggtctgccagcttctgctccgtagcagacaaactgcagtggacgctcgcacagaggacgggaccacacccttgatgctggctgccaggctggcggtggaagacctggttgaagaactgattgcagcccaagcagacgtgggggccagagataaatgggggaaaactgcgctgcactgggctgctgccgtgaacaacgcccgagccgcccgctcgcttctccaggccggagccgataaagatgcccaggacaacagggagcagacgccgctattcctggcggcgcgggaaggagcggtggaagtagcccagctactgctggggctgggggcagcccgagagctgcgggaccaggctgggctagcgccggcggacgtcgctcaccaacgtaaccactgggatctgctgacgctgctggaaggggctgggccaccagaggcccgtcacaaagccacgccgggccgcgaggctgggcccttcccgcgcgcacggacggtgtcagtaagcgtgcccccgcatgggggcggggctctgccgcgctgccggacgctgtcagccggagcaggccctcgtgggggcggagcttgtctgcaggctcggacttggtccgtagacttggctgcgcgggggggcggggcctattctcattgccggagcctctcgggagtaggagcaggaggaggcccgacccctcgcggccgtaggttttctgcaggcatgcgcgggcctcggcccaaccctgcgataatgcgaggaagatacggagtggctgccgggcgcggaggcagggtctcaacggatgactggccctgtgattgggtggccctgggagcttgcggttctgcctccaacattccgatcccgcctccttgccttactccgtccccggagcggggatcacctcaacttgactgtggtcccccagccctccaagaaatgcccataaaccaaggaggagagggtaaaaaatag

SEQ ID NO: 3 - DNA, 23 bp, Artificial sequence
  gRNA
ccctgccagaacgcccagctctg

SEQ ID NO: 4 - DNA, 23 bp, Artificial sequence
  gRNA
cccaccgggcttcgagggccatg

SEQ ID NO: 5 - DNA, 21 bp, Artificial sequence
  Synthetic
gaaggagcccagggtgttatg

SEQ ID NO: 6 - DNA, 22 bp, Artificial sequence
  Synthetic
taggaagagggaccagtgatgt

SEQ ID NO: 7 - DNA, 20 bp, Artificial sequence
  Synthetic
gctagaaacggctccctctg

(19) United States
(12) Patent Application Publication
(10) Pub. No.: US 2020/0022998 A1
(43) Pub. Date: Jan. 23, 2020

(54) MEDICAL USE OF NOTCH4 OR INHIBITORS THEREOF

(71) Applicants: Linzhao Cheng, Columbia, MD (US); BEIJING INSTITUTE OF GENOMICS, CHINESE ACADEMY OF SCIENCES, Beijing (CN)

(72) Inventors: Linzhao Cheng, Columbia, MD (US); Qianfei Wang, Beijing (CN); Yueying Li, Beijing (CN)

(73) Assignees: BEIJING INSTITUTE OF GENOMICS, CHINESE ACADEMY OF SCIENCES, Beijing (CN); Linzhao Cheng, Columbia, MD (US)

(21) Appl. No.: 16/345,600

(22) PCT Filed: Oct. 30, 2017
(86) PCT No.: PCT/CN2017/108411

§ 371 Date: Apr. 26, 2019

(30) Foreign Application Priority Data
Oct. 31, 2016 (CN) 201610928867.2

(51) Int. Cl. A61K 31/713; A61K 38/17; C12N 15/867; C12N 5/078

(52) U.S. Cl. CPC A61K 31/713; A61K 38/177; A61K 45/06; C12N 5/0644; C12N 15/867

(57) ABSTRACT

The invention belongs to the fields of cell biology and medicine, and relates to the medical use of NOTCH4 or the medical use of NOTCH4 inhibitors. In particular, the invention relates to use of NOTCH4 protein or NOTCH4 gene for manufacture of a medicament for promoting hematopoietic stem/progenitor cell differentiation and megakaryocyte differentiation in a mammal, a medicament for treating megakaryocyte dysplasia, or a medicament for promoting platelet production. The invention can effectively promote the production of hematopoietic stem/progenitor cells and megakaryocytes in vitro, thereby significantly improving the efficiency of platelet production in vitro, and having a good application prospect.

### TECHNICAL FIELD

The invention belongs to the fields of cell biology and medicine, and relates to the medical use of NOTCH4 or the medical use of NOTCH4 inhibitors.

### BACKGROUND ART

Megakaryocyte (MK) is a blood cell that produces platelets in the bone marrow, and its main function is to produce platelets. Platelets play an important role in the processes such as hemostasis, wound healing and inflammatory response. Infusion of platelets and/or megakaryocytes in clinic can be used for: thrombocytopenia, post-tumor chemotherapy, surgery, HIV infection, traumatic bleeding, etc. Like other blood cells, megakaryocytes are also differentiated from hematopoietic stein cells (HSCs). Hematopoietic stein cells are differentiated into megakaryocyte-erythroid progenitor cells (MEPs) under relevant stimulation. MEPs are differentiated into megakaryocyte progenitor cells (MKPs) in the presence of a cytokine, thrombopoietin (TPO). Megakaryocyte progenitor cells are proliferated by mitosis, and then megakaryocyte progenitor cells get bigger and bigger by endomitosis, in which the number of chromosomes multiplies constantly, and finally mature megakaryocytes (at most 128N) are formed. On the cell membrane of mature megakaryocytes, a lot of extruding branches are formed, which extend continuously into the blood vessels of sinusoids. These small branches form pre-platelets, and as these branches continue to extend, they gradually break away from megakaryocytes and form platelets.

In current, donated platelets are far from meeting clinical needs due to the reasons mainly including: (1) it is difficult to store platelets in vitro and platelets need to be stored in plasma at 20° C.-24° C. for only 5 days; (2) donors are limited now, and platelets from different donors can not be used in combination; (3) people, who are infused with platelets from other people for several times, are prone to generate antibodies against allogeneic human leukocyte antigen (HLA) and antibodies against human platelet antigen (HPA), and so on. Therefore, it is urgent to develop new technical means for producing platelets or megakaryocytes.

Regeneration of megakaryocytes from stem cells is expected to meet the huge demand in clinic. It has been confirmed by phase I clinical trials conducted in China that transplantation of megakaryocytes produced in vitro is safe and effective. However, the biggest challenge now is the extremely low efficiency for the production of MK in vitro and the production of platelets from MK. At present, good systems for in vitro differentiation of stem cells into megakaryocytes/platelets in the world include: One is from Koji Eto Laboratory, Japan. In 2014, they produced MKs that could be cryopreserved and be passaged for a long time, by exogenous introduction of three factors: c-MYC, BCL-XL and BMI1; however, there was a certain safety risk due to the process of exogenously introducing genes (including the oncogene c-MYC). In the same period, there was a system from Advanced Cell Technology Company, which used the unique medium from this Company and a large number of cytokines, however, its formulation was not disclosed and the process was expensive. The yields are very limited for the above two methods. It needs 100 culture dishes (100 mm) of stem cells and 25 L-50 L medium for culture so as to produce one unit of platelets. The price is very expensive. In addition, in a laboratory in the UK in 2016, megakaryocytes (MKs) were produced from pluripotent stem cells in vitro by exogenous introduction of GATA1, FL11 and TAL1. The MK production increased by 12,500 folds as the one in the previous studies, however, it took 3 months and the efficiency of platelet production from MK was still low.

Notch is a single transmembrane receptor protein of approximately 300 kD, the extracellular portion of which is composed of different numbers of epidermal growth factor-like repeats (EGF-R) and three cysteine-rich LNRs (Lin/Notch repeats), wherein the 11^{th} to 12^{th} repeats of the EGF-R repeat sequence are the key regions for ligand binding. Its intracellular region contains a RAM (RBP-J kappa associated molecular) domain binding to the CSL (CBFl/Suppresor of Hairless/Lag1) transcription factor, 6 ankyrin (cdc10/ankyrin, ANK) repeats, 2 nuclear localization signals (NLS) and 1 PEST domain associated with the degradation of intracellular segment of Notch protein. In mammals, Notch receptors can be classified into four types: Notch1, Notch2, Notch3, and Notch4. There is a significant difference among the four receptors of Notch pathway. For example, Notch1 and Notch2 receptors contain 36 EGF repeats, while Notch3 and Notch4 contain 34 and 29 EGF repeats, respectively; Notch receptors 1-3 contain two nuclear localization signals (NLS), while Notch4 receptor contains one nuclear localization signal (NLS). In addition, the intracellular transcription activation domain (TAD) of Notch1 receptor is more active than that of Notch2, however, there is no TAD in Notch3 and Notch4. Notch receptors 1-4 are also different from each other in terms of tissue specificity. The four receptor proteins of Notch pathway are different from each other in terms of distribution and expression level in the blood system, pancreas, liver, lung, cardiovascular system, etc. Notch receptor protein is synthesized and processed, and then is bound to Notch ligand of adjacent cells so as to initiate signal transduction pathway. It is found that there are five kinds of human Notch ligands, i.e. Dll1, Dll3, Dll4, Jag1 and Jag2. When the ligand binds to the extracellular domain of Notch receptor protein, the Notch protein is cleaved by TNF-α-converting enzyme (TACE) and γ-secretase successively, to release the intracellular domain (ICN) of Notch into the nucleus, and the intracellular domain (ICN) of Notch is bound to CSL transcription factor to form a complex, thereby promoting the expression of target genes such as HES, and further playing a biological role. Notch receptor, Notch ligand, CSL-DNA binding protein constitute a complete Notch signaling pathway. Notch signaling pathway is widely present on the cell surface, mediates cell-to-cell signaling, regulates cell transcription, and affects the proliferation and differentiation of cells. During the development of vertebrates and invertebrates, it plays an important role in deciding the cell fate. Notch signaling pathway is involved in the regulation of nervous system development, organ development, immune system formation and tumor formation; and regulation of cell differentiation and tissue formation. In addition, Notch receptor is highly expressed in hematopoietic progenitor cells, while Notch ligand is highly expressed in bone marrow stromal cells, and Notch signaling pathway plays an important role in the self-renewal and differentiation of hematopoietic stem/progenitor cells.

A paper published in Cell Stein Cell in 2008 revealed that Notch4 was expressed in the highest level in megakaryocyte-erythroid progenitor cells, and played a positive regulatory role in megakaryocyte differentiation in mice (Mercher T, Cornejo M G, Sears C, Kindler T, Moore S A, Maillard I, Pear W S, Aster J C, Gilliland D G, Notch signaling specifies megakaryocyte development from hematopoietic stein cells, Cell Stein Cell. 2008 Sep. 11; 3(3):314-26.).

Now, there is an urgent need to develop new technical means for promoting the production of megakaryocytes and/or megakaryocyte progenitor cells in a mammal or for promoting platelet production.

Contents of Invention

By conducting deep research and creative work, the inventor found the inhibitory effect of NOTCH4 protein or NOTCH4 gene on differentiation and production of human hematopoietic stem/progenitor cells and megakaryocytes; and the inventor surprisingly found that down-regulation of the expression level of NOTCH4 gene, or inhibition of Notch pathway, could significantly promote the production of hematopoietic stem/progenitor cells and megakaryocytes in vitro, enhance the proportion or number of megakaryocytes produced by differentiation of stein cells in vitro, and could be used for promoting the re-generation of megakaryocytes or platelets in vitro. Thus, the invention is provided as followed.

In an aspect, the invention relates to use of any one of the following items (1) to (5) for manufacture of a medicament for modulating (e.g. promoting or inhibiting) the production of hematopoietic stem/progenitor cells and/or megakaryocytes and/or megakaryocyte progenitor cells in a mammal, a medicament for treating megakaryocyte dysplasia, or a medicament for modulating (e.g. promoting or inhibiting) platelet production:

(1) NOTCH4 protein:

(2) NOTCH4 gene;

(3) a nucleic acid construct, comprising a polynucleotide for completely or partially knocking out NOTCH4 gene; preferably, the polynucleotide is siRNA such as shRNA, or is a guide RNA for use in CRISPR/Cas9 system; preferably, the sequence of the guide RNA is set forth in SEQ ID NO: 3 and/or SEQ ID NO: 4.

preferably, the nucleic acid construct is a recombinant vector, preferably a recombinant expression vector, more preferably a recombinant lentiviral expression vector; and

(4) a host cell, in which NOTCH4 gene is completely or partially knocked out; preferably, comprising the nucleic acid construct according to the item (3); and

(5) a composition, comprising any one of the preceding items (1) to (4).

In another aspect, the invention relates to use of any one of the following items \textcircled{1} to \textcircled{4} for manufacture of a medicament for promoting the production of hematopoietic stem/progenitor cells and/or megakaryocytes and/or megakaryocyte progenitor cells in a mammal, a medicament for treating megakaryocyte dysplasia, or a medicament for promoting platelet production:

\textcircled{1} a drug for inhibiting or reducing the expression of NOTCH4 gene;

\textcircled{2} a drug for inhibiting or blocking the activity of NOTCH4 protein;

\textcircled{3} a drug for completely or partially knocking out NOTCH4 gene; and

\textcircled{4} a Notch pathway inhibitor such as a tumor necrosis factor-α-converting enzyme inhibitor or a γ-secretase inhibitor.

In an embodiment of the invention, in the use, the mammal is a primate, such as a human.

In an embodiment of the invention, in the use, the γ-secretase inhibitor is one or more selected from the group consisting of RO4929097, L-685458, LY411575, PF-03084014, YO-01027, DAPT and FLI-06.

In a particular embodiment of the invention, the inventor employed an in vitro differentiation system of stem cells, to screen Notch pathway inhibitors, including γ-secretase inhibitors and antibodies against NOTCH4, etc., wherein the γ-secretase inhibitors (RO4929097, L685458 , DAPT, etc.) and antibodies against NOTCH4 could significantly promote the production of megakaryocyte progenitor cells and/or mature megakaryocytes in vitro.

In an embodiment of the invention, in the use, the drug for inhibiting or blocking the activity of NOTCH4 protein is an antibody against NOTCH4 protein; preferably, the antibody is a monoclonal antibody.

In an embodiment of the invention, in the use, the drug for completely or partially knocking out NOTCH4 gene is a polynucleotide for completely or partially knocking out NOTCH4 gene; preferably, the polynucleotide is siRNA such as shRNA, or is a guide RNA for use in CRISPR/Cas9 system; preferably, the sequence of the guide RNA is set forth in SEQ ID NO: 3 and/or SEQ ID NO: 4.

The invention further relates to a recombinant vector, comprising a polynucleotide for completely or partially knocking out NOTCH4 gene; preferably, the polynucleotide is siRNA such as shRNA, or is a guide RNA for use in CRISPR/Cas9 system; preferably, the recombinant vector is a recombinant lentiviral vector; preferably, the sequence of the guide RNA is set forth in SEQ ID NO: 3 and/or SEQ ID NO: 4.

In an embodiment of the invention, the recombinant vector, is used for modulating (e.g. promoting or inhibiting) the production of hematopoietic stem/progenitor cells and/or megakaryocytes and/or megakaryocyte progenitor cells in a mammal, for treating megakaryocyte dysplasia, for modulating (e.g. promoting or inhibiting) platelet production, for producing megakaryocytes and/or megakaryocyte progenitor cells in vitro, for producing platelets in vitro, or for screening a medicament for modulating (e.g. promoting or inhibiting) the production of megakaryocytes and/or megakaryocyte progenitor cells in a mammal, a medicament for treating megakaryocyte dysplasia, or a medicament for modulating (e.g. promoting or inhibiting) platelet production.

The invention further relates to a host cell, comprising the recombinant vector according to the invention, or in which NOTCH4 gene is completely or partially knocked out;

preferably, the host cell is an embryonic stein cell, an induced pluripotent stein cell or a hematopoietic stem/progenitor cell;

preferably, the induced pluripotent stein cell is a recombinant BC1 cell or a recombinant Aicas9 cell.

In an embodiment of the invention, the host cell is used for modulating (e.g. promoting or inhibiting) the production of hematopoietic stem/progenitor cells and/or megakaryocytes and/or megakaryocyte progenitor cells in a mammal, for treating megakaryocyte dysplasia, for modulating (e.g. promoting or inhibiting) platelet production, for producing megakaryocytes and/or megakaryocyte progenitor cells in vitro, for producing platelets in vitro, or for screening a medicament for modulating (e.g. promoting or inhibiting) the production of megakaryocytes and/or megakaryocyte progenitor cells in a mammal, a medicament for treating megakaryocyte dysplasia, or a medicament for modulating (e.g. promoting or inhibiting) platelet production. The invention further relates to a composition, comprising:

the host cell according to the invention, and cell culture medium.

In another aspect, the invention relates to a kit, comprising individually packaged embryonic stein cells, induced pluripotent stein cells or hematopoietic stem/progenitor cells, and a drug or an agent selected from the group consisting of the following items 1) to 3):

1) a drug for inhibiting or reducing the expression of NOTCH4 gene;

2) a drug for inhibiting or blocking the activity of NOTCH4 protein;

3) a drug for completely or partially knocking out NOTCH4 gene; and

4) a Notch pathway inhibitor such as a tumor necrosis factor-α-converting enzyme inhibitor or a γ-secretase inhibitor;

preferably, the induced pluripotent stem cell is a recombinant BC1 cell or a recombinant Aicas9 cell.

In an embodiment of the invention, in the kit, the γ-secretase inhibitor is one or more selected from the group consisting of RO4929097, L-685458, LY411575, PF-03084014, YO-01027, DAPT and FLI-06.

In an embodiment of the invention, in the kit, the drug for inhibiting or blocking the activity of NOTCH4 protein is an antibody against NOTCH4 protein; preferably, the antibody is a monoclonal antibody.

In an embodiment of the invention, in the kit, the drug for completely or partially knocking out NOTCH4 gene is a polynucleotide for completely or partially knocking out NOTCH4 gene; preferably, the polynucleotide is siRNA such as shRNA, or is a guide RNA for use in CRISPR/Cas9 system; preferably, the sequence of the guide RNA is set forth in SEQ ID NO: 3 and/or SEQ ID NO: 4.

In an embodiment of the invention, the kit is used for modulating (e.g. promoting or inhibiting) the production of hematopoietic stem/progenitor cells and/or megakaryocytes and/or megakaryocyte progenitor cells in a mammal, for treating megakaryocyte dysplasia, for modulating (e.g. promoting or inhibiting) platelet production, for producing megakaryocytes and/or megakaryocyte progenitor cells in vitro, for producing platelets in vitro, or for screening a medicament for modulating (e.g. promoting or inhibiting) the production of megakaryocytes and/or megakaryocyte progenitor cells in a mammal, a medicament for treating megakaryocyte dysplasia, or a medicament for modulating (e.g. promoting or inhibiting) platelet production.

In another aspect, the invention relates to a method for producing hematopoietic stem/progenitor cells and/or megakaryocytes and/or megakaryocyte progenitor cells in vitro, comprising:

the step of inhibiting or reducing the expression of NOTCH4 gene in an embryonic stem cell, an induced pluripotent stem cell or a hematopoietic stem/progenitor cell; or

the step of inhibiting or blocking the activity of NOTCH4 protein in an embryonic stem cell, an induced pluripotent stem cell or a hematopoietic stem/progenitor cell.

In an embodiment of the invention, “inhibiting or reducing the expression of NOTCH4 gene in an embryonic stem cell, an induced pluripotent stem cell or a hematopoietic stem/progenitor cell” or “inhibiting or blocking the activity of NOTCH4 protein in an embryonic stem cell, an induced pluripotent stem cell or a hematopoietic stem/progenitor cell”, is achieved by using an effective amount of the composition according to the invention or the kit according to the invention; or by adding an effective amount of any one of the following items \textcircled{1} to \textcircled{4}:

\textcircled{1} a drug for inhibiting or reducing the expression of NOTCH4 gene;

\textcircled{2} a drug for inhibiting or blocking the activity of NOTCH4 protein;

\textcircled{3} a drug for completely or partially knocking out NOTCH4 gene; and

\textcircled{4} a Notch pathway inhibitor such as a tumor necrosis factor-α-converting enzyme inhibitor or a γ-secretase inhibitor.

In another aspect, the invention relates to a method for producing platelets in vitro, comprising:

the step of inhibiting or reducing the expression of NOTCH4 gene in an embryonic stem cell, an induced pluripotent stem cell or a hematopoietic stem/progenitor cell; or

the step of inhibiting or blocking the activity of NOTCH4 protein in an embryonic stem cell, an induced pluripotent stem cell or a hematopoietic stem/progenitor cell.

In an embodiment of the invention, “inhibiting or reducing the expression of NOTCH4 gene in an embryonic stem cell, an induced pluripotent stem cell or a hematopoietic stem/progenitor cell” or “inhibiting or blocking the activity of NOTCH4 protein in an embryonic stem cell, an induced pluripotent stem cell or a hematopoietic stem/progenitor cell”, is achieved by using an effective amount of the composition according to the invention or the kit according to the invention; or by adding an effective amount of any one of the following items \textcircled{1} to \textcircled{4}:

\textcircled{1} a drug for inhibiting or reducing the expression of NOTCH4 gene;

\textcircled{2} a drug for inhibiting or blocking the activity of NOTCH4 protein;

\textcircled{3} a drug for completely or partially knocking out NOTCH4 gene; and

\textcircled{4} a Notch pathway inhibitor such as a tumor necrosis factor-α-converting enzyme inhibitor or a γ-secretase inhibitor.

In another aspect, the invention relates to a method for screening a medicament for modulating (e.g. promoting or inhibiting) the production of hematopoietic stem/progenitor cells and/or megakaryocytes and/or megakaryocyte progenitor cells in a mammal, a medicament for treating megakaryocyte dysplasia, or a medicament for modulating (e.g. promoting or inhibiting) platelet production, comprising:

the step of detecting a test medicament for its inhibition or reduction of the expression level of NOTCH4 gene in an embryonic stem cell, an induced pluripotent stem cell or a hematopoietic stem/progenitor cell; or

the step of detecting a test medicament for its inhibition or blockage of the activity level of NOTCH4 protein in an embryonic stem cell, an induced pluripotent stem cell or a hematopoietic stem/progenitor cell.

If the test medicament can inhibit or reduce the expression level of NOTCH4 gene, or inhibit or block the activity level of NOTCH4 protein, it can be used as a candidate medicament.

In an embodiment of the invention, the test medicament is added to an isolated embryonic stem cell, an induced pluripotent stem cell or a hematopoietic stem/progenitor cell, and the corresponding cell without the addition of the test medicament is used as control.

In an embodiment of the invention, the test medicament is administered to a mammal such as a human or a mouse, to observe or detect whether the symptom or index of interest is improved.

The invention further relates to any one of the following items (1) to (5), for use in manufacture of a medicament for modulating (e.g. promoting or inhibiting) the production of hematopoietic stem/progenitor cells and/or megakaryocytes and/or megakaryocyte progenitor cells in a mammal, a medicament for treating megakaryocyte dysplasia, or a medicament for modulating (e.g. promoting or inhibiting) platelet production:

(1) NOTCH4 protein:

(2) NOTCH4 gene;

(3) a nucleic acid construct, comprising a polynucleotide for completely or partially knocking out NOTCH4 gene; preferably, the polynucleotide is siRNA such as shRNA, or is a guide RNA for use in CRISPR/Cas9 system;

preferably, the nucleic acid construct is a recombinant vector, preferably a recombinant expression vector, more preferably a recombinant lentiviral expression vector;

(4) a host cell, in which NOTCH4 gene is completely or partially knocked out; preferably, the host cell comprises the nucleic acid construct according to the item (3); and

(5) a composition, comprising any one of the preceding items (1) to (4);

preferably, the mammal is a primate, such as a human.

The invention further relates to a drug selected from any one of the following items \textcircled{1} to \textcircled{4}, for use in promoting the production of hematopoietic stem/progenitor cells and/or megakaryocytes and/or megakaryocyte progenitor cells in a mammal, treating megakaryocyte dysplasia or promoting platelet production:

\textcircled{1} a drug for inhibiting or reducing the expression of NOTCH4 gene;

\textcircled{2} a drug for inhibiting or blocking the activity of NOTCH4 protein;

\textcircled{3} a drug for completely or partially knocking out NOTCH4 gene; and

\textcircled{4} a Notch pathway inhibitor such as a tumor necrosis factor-α-converting enzyme inhibitor or a γ-secretase inhibitor.

In an embodiment of the invention, the mammal is a primate, such as a human.

In an embodiment of the invention, the γ-secretase inhibitor is one or more selected from the group consisting of RO4929097, L-685458, LY411575, PF-03084014, YO-01027, DAPT and FLI-06.

In an embodiment of the invention, the drug for inhibiting or blocking the activity of NOTCH4 protein is an antibody against NOTCH4 protein; preferably, the antibody is a monoclonal antibody.

In an embodiment of the invention, the drug for completely or partially knocking out NOTCH4 gene is a polynucleotide for completely or partially knocking out NOTCH4 gene; preferably, the polynucleotide is siRNA such as shRNA, or is a guide RNA for use in CRISPR/Cas9 system; preferably, the sequence of the guide RNA is set forth in SEQ ID NO: 3 and/or SEQ ID NO: 4.

The invention further relates to a method for treating and/or preventing megakaryocyte dysplasia or for treating and/or preventing a disease associated with abnormal platelet (e.g. thrombocytopenia), comprising the step of administering to a subject an effective amount of the host cell, composition or kit according to the invention, or comprising the step of administering to a subject an effective amount of any one of the following items \textcircled{1} to \textcircled{4}:

\textcircled{1} a drug for inhibiting or reducing the expression of NOTCH4 gene;

\textcircled{2} a drug for inhibiting or blocking the activity of NOTCH4 protein;

\textcircled{3} a drug for completely or partially knocking out NOTCH4 gene; and

\textcircled{4} a Notch pathway inhibitor such as a tumor necrosis factor-α-converting enzyme inhibitor or a γ-secretase inhibitor.

In an embodiment of the invention, in the method, the γ-secretase inhibitor is one or more selected from the group consisting of RO4929097, L-685458, LY411575, PF-03084014, YO-01027, DAPT and FLI-06.

In an embodiment of the invention, in the method, the drug for inhibiting or blocking the activity of NOTCH4 protein is an antibody against NOTCH4 protein; preferably, the antibody is a monoclonal antibody.

In an embodiment of the invention, in the method, the drug for completely or partially knocking out NOTCH4 gene is a polynucleotide for completely or partially knocking out NOTCH4 gene; preferably, the polynucleotide is siRNA such as shRNA, or is a guide RNA for use in CRISPR/Cas9 system; preferably, the sequence of the guide RNA is set forth in SEQ ID NO: 3 and/or SEQ ID NO: 4.

Inhibiting the activity level of NOTCH4 protein in a subject or down-regulating of the expression level of NOTCH4 gene in a subject, depends on a lot of factors, such as the severity of a disease to be treated, the gender, age, weight and individual response of a patient or an animal, as well as the condition and medical history of a patient to be treated. The method generally used in the art is to start the dose from a level lower than the one needed for the desired therapeutic effect and/or preventive effect, and is gradually increased until the desired effect is achieved.

In the invention, the term “megakaryocyte differentiation” refers to in vitro megakaryocyte differentiation, wherein in the specific process, embryonic stem cells/induced pluripotent stem cells capable of multi-directional differentiation are differentiated into hematopoietic stem/progenitor cells in the presence of a combination of various cytokines, and then are differentiated into megakaryocyte progenitor cells (MKP) and mature megakaryocytes in the presence of a cytokine, thrombopoietin (TPO).

In the invention, when the amino acid sequence of NOTCH4 protein or Notch4 receptor is mentioned, it includes full-length NOTCH4 protein, and a fusion protein thereof. However, a person skilled in the art understands that mutation or variation (including, but not limited to substitution, deletion and/or addition) may occur naturally or be introduced artificially in the amino acid sequence of NOTCH4 protein, without affecting its biological function. In an embodiment of the invention, NOTCH4 protein is human NOTCH4 protein.

Human NOTCH4 protein has an amino acid sequence of: (2003 AA)

(SEQ ID NO: 1)
MQPPSLLLLLLLLLLLCVSVVRPRGLLCGSFPEPCANGGTCLSLSLGQGT
CQCAPGFLGETCQFPDPCQNAQLCQNGGSCQALLPAPLGLPSSPSPLTPS
FLCTCLPGFTGERCQAKLEDPCPPSFCSKRGRCHIQASGRPQCSCMPGWT
GEQCQLRDFCSANPCVNGGVCLATYPQIQCHCPPGFEGHACERDVNECFQ
DPGPCPKGTSCHNTLGSFQCLCPVGQEGPRCELRAGPCPPRGCSNGGTCQ
LMPEKDSTFHLCLCPPGFIGPDCEVNPDNCVSHQCQNGGTCQDGLDTYTC
LCPETWTGWDCSEDVDECETQGPPHCRNGGTCQNSAGSFHCVCVSGWGGT
SCEENLDDCIAATCAPGSTCIDRVGSFSCLCPPGRTGLLCHLEDMCLSQP
CHGDAQCSTNPLTGSTLCLCQPGYSGPTCHQDLDECLMAQQGPSPCEHGG
SCLNTPGSFNCLCPPGYTGSRCEADHNECLSQPCHPGSTCLDLLATFHCL
CPPGLEGQLCEVETNECASAPCLNHADCHDLLNGFQCICLPGFSGTRCEE
DIDECRSSPCANGGQCQDQPGAFHCKCLPGFEGPRCQTEVDECLSDPCPV
GASCLDLPGAFFCLCPSGFTGQLCEVPLCAPNLCQPKQICKDQKDKANCL
CPDGSPGCAPPEDNCTCHHGHCQRSSCVCDVGWTGPECEAELGGCISAPC
AHGGTCYPQPSGYNCTCPTGYTGPTCSEEMTACHSGPCLNGGSCNPSPGG
YYCTCPPSHTGPQCQTSTDYCVSAPCFNGGTCVNRPGTFSCLCAMGFQGP
RCEGKLRPSCADSPCRNRATCQDSPQGPRCLCPTGYTGGSCQTLMDLCAQ
KPCPRNSHCLQTGPSFHCLCLQGWTGPLCNLPLSSCQKAALSQGIDVSSL
CHNGGLCVDSGPSYFCHCPPGFQGSLCQDHVNPCESRPCQNGATCMAQPS
GYLCQCAPGYDGQNCSKELDACQSQPCHNHGTCTPKPGGFHCACPPGFVG
LRCEGDVDECLDQPCHPTGTAACHSLANAFYCQCLPGHTGQWCEVEIDPC
HSQPCFHGGTCEATAGSPLGFICHCPKGFEGPTCSHRAPSCGFHHCHHGG
LCLPSPKPGFPPRCACLSGYGGPDCLTPPAPKGCGPPSPCLYNGSCSETT
GLGGPGFRCSCPHSSPGPRCQKPGAKGCEGRSGDGACDAGCSGPGGNWDG
GDCSLGVPDPWKGCPSHSRCWLLFRDGQCHPQCDSEECLFDGYDCETPPA
CTPAYDQYCHDHFHNGHCEKGCNTAECGWDGGDCRPEDGDPEWGPSLALL
VVLSPPALDQQLFALARVLSLTLRVGLWVRKDRDGRDMVYPYPGARAEEK
LGGTRDPTYQERAAPQTQPLGKETDSLSAGFVVVMGVDLSRCGPDHPASR
CPWDPGLLLRFLAAMAAVGALEPLLPGPLLAVHPHAGTAPPANQLPWPVL
CSPVAGVILLALGALLVLQLIRRRRREHGALWLPPGFTRRPRTQSAPHRR
RPPLGEDSIGLKALKPKAEVDEDGVVMCSGPEEGEEVGQAEETGPPSTCQ
LWSLSGGCGALPQAAMLTPPQESEMEAPDLDTRGPDGVTPLMSAVCCGEV
QSGTFQGAWLGCPEPWEPLLDGGACPQAHTVGTGETPLHLAARFSRPTAA
RRLLEAGANPNQPDRAGRTPLHAAVAADAREVCQLLLRSRQTAVDARTED
GTTPLMLAARLAVEDLVEELIAAQADVGARDKWGKTALHWAAAVNNARAA
RSLLQAGADKDAQDNREQTPLFLAAREGAVEVAQLLLGLGAARELRDQAG
LAPADVAHQRNHWDLLTLLEGAGPPEARHKATPGREAGPFPRARTVSVSV
PPHGGGALPRCRTLSAGAGPRGGGACLQARTWSVDLAARGGGAYSHCRSL
SGVGAGGGPTPRGRRFSAGMRGPRPNPAIMRGRYGVAAGRGGRVSTDDWP
CDWVALGACGSASNIPIPPPCLTPSPERGSPQLDCGPPALQEMPINQGGE
GKK

The nucleotide sequence encoding human NOTCH4 protein (6012 bp)

(SEQ ID NO: 2)
ATGCAGCCCCCTTCACTGCTGCTGCTGCTGCTGCTGCTGCTGCTGCTATG
TGTCTCAGTGGTCAGACCCAGAGGGCTGCTGTGTGGGAGTTTCCCAGAAC
CCTGTGCCAATGGAGGCACCTGCCTGAGCCTGTCTCTGGGACAAGGGACC
TGCCAGTGTGCCCCTGGCTTCCTGGGTGAGACGTGCCAGTTTCCTGACCC
CTGCCAGAACGCCCAGCTCTGCCAAAATGGAGGCAGCTGCCAAGCCCTGC
TTCCCGCTCCCCTAGGGCTCCCCAGCTCTCCCTCTCCATTGACACCCAGC
TTCTTGTGCACTTGCCTCCCTGGCTTCACTGGTGAGAGATGCCAGGCCAA
GCTTGAAGACCCTTGTCCTCCCTCCTTCTGTTCCAAAAGGGGCCGCTGCC
ACATCCAGGCCTCGGGCCGCCCACAGTGCTCCTGCATGCCTGGATGGACA
GGTGAGCAGTGCCAGCTTCGGGACTTCTGTTCAGCCAACCCATGTGTTAA
TGGAGGGGTGTGTCTGGCCACATACCCCCAGATCCAGTGCCACTGCCCAC
CGGGCTTCGAGGGCCATGCCTGTGAACGTGATGTCAACGAGTGCTTCCAG
GACCCAGGACCCTGCCCCAAAGGCACCTCCTGCCATAACACCCTGGGCTC
CTTCCAGTGCCTCTGCCCTGTGGGGCAGGAGGGTCCACGTTGTGAGCTGC
GGGCAGGACCCTGCCCTCCTAGGGGCTGTTCGAATGGGGGCACCTGCCAG
CTGATGCCAGAGAAAGACTCCACCTTTCACCTCTGCCTCTGTCCCCCAGG
TTTCATAGGCCCAGACTGTGAGGTGAATCCAGACAACTGTGTCAGCCACC
AGTGTCAGAATGGGGGCACTTGCCAGGATGGGCTGGACACCTACACCTGC
CTCTGCCCAGAAACCTGGACAGGCTGGGACTGCTCCGAAGATGTGGATGA
GTGTGAGACCCAGGGTCCCCCTCACTGCAGAAACGGGGGCACCTGCCAGA
ACTCTGCTGGTAGCTTTCACTGCGTGTGTGTGAGTGGCTGGGGCGGCACA
AGCTGTGAGGAGAACCTGGATGACTGTATTGCTGCCACCTGTGCCCCGGG
ATCCACCTGCATTGACCGGGTGGGCTCTTTCTCCTGCCTCTGCCCACCTG
GACGCACAGGACTCCTGTGCCACTTGGAAGACATGTGTCTGAGCCAGCCG
TGCCATGGGGATGCCCAATGCAGCACCAACCCCCTCACAGGCTCCACACT
CTGCCTGTGTCAGCCTGGCTATTCGGGGCCCACCTGCCACCAGGACCTGG
ACGAGTGTCTGATGGCCCAGCAAGGCCCAAGTCCCTGTGAACATGGCGGT
TCCTGCCTCAACACTCCTGGCTCCTTCAACTGCCTCTGTCCACCTGGCTA
CACAGGCTCCCGTTGTGAGGCTGATCACAATGAGTGCCTCTCCCAGCCCT
GCCACCCAGGAAGCACCTGTCTGGACCTACTTGCCACCTTCCACTGCCTC
TGCCCGCCAGGCTTAGAAGGGCAGCTCTGTGAGGTGGAGACCAACGAGTG
TGCCTCAGCTCCCTGCCTGAACCACGCGGATTGCCATGACCTGCTCAACG
GCTTCCAGTGCATCTGCCTGCCTGGATTCTCCGGCACCCGATGTGAGGAG
GATATCGATGAGTGCAGAAGCTCTCCCTGTGCCAATGGTGGGCAGTGCCA
GGACCAGCCTGGAGCCTTCCACTGCAAGTGTCTCCCAGGCTTTGAAGGGC
CACGCTGTCAAACAGAGGTGGATGAGTGCCTGAGTGACCCATGTCCCGTT
GGAGCCAGCTGCCTTGATCTTCCAGGAGCCTTCTTTTGCCTCTGCCCCTC
TGGTTTCACAGGCCAGCTCTGTGAGGTTCCCCTGTGTGCTCCCAACCTGT
GCCAGCCCAAGCAGATATGTAAGGACCAGAAAGACAAGGCCAACTGCCTC
TGTCCTGATGGAAGCCCTGGCTGTGCCCCACCTGAGGACAACTGCACCTG
CCACCACGGGCACTGCCAGAGATCCTCATGTGTGTGTGACGTGGGTTGGA
CGGGGCCAGAGTGTGAGGCAGAGCTAGGGGGCTGCATCTCTGCACCCTGT
GCCCATGGGGGGACCTGCTACCCCCAGCCCTCTGGCTACAACTGCACCTG
CCCTACAGGCTACACAGGACCCACCTGTAGTGAGGAGATGACAGCTTGTC
ACTCAGGGCCATGTCTCAATGGCGGCTCCTGCAACCCTAGCCCTGGAGGC
TACTACTGCACCTGCCCTCCAAGCCACACAGGGCCCCAGTGCCAAACCAG
CACTGACTACTGTGTGTCTGCCCCGTGCTTCAATGGGGGTACCTGTGTGA
ACAGGCCTGGCACCTTCTCCTGCCTCTGTGCCATGGGCTTCCAGGGCCCG
CGCTGTGAGGGAAAGCTCCGCCCCAGCTGTGCAGACAGCCCCTGTAGGAA
TAGGGCAACCTGCCAGGACAGCCCTCAGGGTCCCCGCTGCCTCTGCCCCA
CTGGCTACACCGGAGGCAGCTGCCAGACTCTGATGGACTTATGTGCCCAG
AAGCCCTGCCCACGCAATTCCCACTGCCTCCAGACTGGGCCCTCCTTCCA
CTGCTTGTGCCTCCAGGGATGGACCGGGCCTCTCTGCAACCTTCCACTGT
CCTCCTGCCAGAAGGCTGCACTGAGCCAAGGCATAGACGTCTCTTCCCTT
TGCCACAATGGAGGCCTCTGTGTCGACAGCGGCCCCTCCTATTTCTGCCA
CTGCCCCCCTGGATTCCAAGGCAGCCTGTGCCAGGATCACGTGAACCCAT
GTGAGTCCAGGCCTTGCCAGAACGGGGCCACCTGCATGGCCCAGCCCAGT
GGGTATCTCTGCCAGTGTGCCCCAGGCTACGATGGACAGAACTGCTCAAA
GGAACTCGATGCTTGTCAGTCCCAACCCTGTCACAACCATGGAACCTGTA
CTCCCAAACCTGGAGGATTCCACTGTGCCTGCCCTCCAGGCTTTGTGGGG
CTACGCTGTGAGGGAGACGTGGACGAGTGTCTGGACCAGCCCTGCCACCC
CACAGGCACTGCAGCCTGCCACTCTCTGGCCAATGCCTTCTACTGCCAGT
GTCTGCCTGGACACACAGGCCAGTGGTGTGAGGTGGAGATAGACCCCTGC
CACAGCCAACCCTGCTTTCATGGAGGGACCTGTGAGGCCACAGCAGGATC
ACCCCTGGGTTTCATCTGCCACTGCCCCAAGGGTTTTGAAGGCCCCACCT
GCAGCCACAGGGCCCCTTCCTGCGGCTTCCATCACTGCCACCACGGAGGC
CTGTGTCTGCCCTCCCCTAAGCCAGGCTTCCCACCACGCTGTGCCTGCCT
CAGTGGCTATGGGGGTCCTGACTGCCTGACCCCACCAGCTCCTAAAGGCT
GTGGCCCTCCCTCCCCATGCCTATACAATGGCAGCTGCTCAGAGACCACG
GGCTTGGGGGGCCCAGGCTTTCGATGCTCCTGCCCTCACAGCTCTCCAGG
GCCCCGGTGTCAGAAACCCGGAGCCAAGGGGTGTGAGGGCAGAAGTGGAG
ATGGGGCCTGCGATGCTGGCTGCAGTGGCCCGGGAGGAAACTGGGATGGA
GGGGACTGCTCTCTGGGAGTCCCAGACCCCTGGAAGGGCTGCCCCTCCCA
CTCTCGGTGCTGGCTTCTCTTCCGGGACGGGCAGTGCCACCCACAGTGTG
ACTCTGAAGAGTGTCTGTTTGATGGCTACGACTGTGAGACCCCTCCAGCC
TGCACTCCAGCCTATGACCAGTACTGCCATGATCACTTCCACAACGGGCA
CTGTGAGAAAGGCTGCAACACTGCAGAGTGTGGCTGGGATGGAGGTGACT
GCAGGCCTGAAGATGGGGACCCAGAGTGGGGGCCCTCCCTGGCCCTGCTG
GTGGTACTGAGCCCCCCAGCCCTAGACCAGCAGCTGTTTGCCCTGGCCCG
GGTGCTGTCCCTGACTCTGAGGGTAGGACTCTGGGTAAGGAAGGATCGTG
ATGGCAGGGACATGGTGTACCCCTATCCTGGGGCCCGGGCTGAAGAAAAG
CTAGGAGGAACTCGGGACCCCACCTATCAGGAGAGAGCAGCCCCTCAAAC
GCAGCCCCTGGGCAAGGAGACCGACTCCCTCAGTGCTGGGTTTGTGGTGG
TCATGGGTGTGGATTTGTCCCGCTGTGGCCCTGACCACCCGGCATCCCGC
TGTCCCTGGGACCCTGGGCTTCTACTCCGCTTCCTTGCTGCGATGGCTGC
AGTGGGAGCCCTGGAGCCCCTGCTGCCTGGACCACTGCTGGCTGTCCACC
CTCATGCAGGGACCGCACCCCCTGCCAACCAGCTTCCCTGGCCTGTGCTG
TGCTCCCCAGTGGCCGGGGTGATTCTCCTGGCCCTAGGGGCTCTTCTCGT
CCTCCAGCTCATCCGGCGTCGACGCCGAGAGCATGGAGCTCTCTGGCTGC
CCCCTGGTTTCACTCGACGGCCTCGGACTCAGTCAGCTCCCCACCGACGC
CGGCCCCCACTAGGCGAGGACAGCATTGGTCTCAAGGCACTGAAGCCAAA
GGCAGAAGTTGATGAGGATGGAGTTGTGATGTGCTCAGGCCCTGAGGAGG
GAGAGGAGGTGGGCCAGGCTGAAGAAACAGGCCCACCCTCCACGTGCCAG
CTCTGGTCTCTGAGTGGTGGCTGTGGGGCGCTCCCTCAGGCAGCCATGCT
AACTCCTCCCCAGGAATCTGAGATGGAAGCCCCTGACCTGGACACCCGTG
GACCTGATGGGGTGACACCCCTGATGTCAGCAGTTTGCTGTGGGGAAGTA
CAGTCCGGGACCTTCCAAGGGGCATGGTTGGGATGTCCTGAGCCCTGGGA
ACCTCTGCTGGATGGAGGGGCCTGTCCCCAGGCTCACACCGTGGGCACTG
GGGAGACCCCCCTGCACCTGGCTGCCCGATTCTCCCGGCCAACCGCTGCC
CGCCGCCTCCTTGAGGCTGGAGCCAACCCCAACCAGCCAGACCGGGCAGG
GCGCACACCCCTTCATGCTGCTGTGGCTGCTGATGCTCGGGAGGTCTGCC
AGCTTCTGCTCCGTAGCAGACAAACTGCAGTGGACGCTCGCACAGAGGAC
GGGACCACACCCTTGATGCTGGCTGCCAGGCTGGCGGTGGAAGACCTGGT
TGAAGAACTGATTGCAGCCCAAGCAGACGTGGGGGCCAGAGATAAATGGG
GGAAAACTGCGCTGCACTGGGCTGCTGCCGTGAACAACGCCCGAGCCGCC
CGCTCGCTTCTCCAGGCCGGAGCCGATAAAGATGCCCAGGACAACAGGGA
GCAGACGCCGCTATTCCTGGCGGCGCGGGAAGGAGCGGTGGAAGTAGCCC
AGCTACTGCTGGGGCTGGGGGCAGCCCGAGAGCTGCGGGACCAGGCTGGG
CTAGCGCCGGCGGACGTCGCTCACCAACGTAACCACTGGGATCTGCTGAC
GCTGCTGGAAGGGGCTGGGCCACCAGAGGCCCGTCACAAAGCCACGCCGG
GCCGCGAGGCTGGGCCCTTCCCGCGCGCACGGACGGTGTCAGTAAGCGTG
CCCCCGCATGGGGGCGGGGCTCTGCCGCGCTGCCGGACGCTGTCAGCCGG
AGCAGGCCCTCGTGGGGGCGGAGCTTGTCTGCAGGCTCGGACTTGGTCCG
TAGACTTGGCTGCGCGGGGGGGCGGGGCCTATTCTCATTGCCGGAGCCTC
TCGGGAGTAGGAGCAGGAGGAGGCCCGACCCCTCGCGGCCGTAGGTTTTC
TGCAGGCATGCGCGGGCCTCGGCCCAACCCTGCGATAATGCGAGGAAGAT
ACGGAGTGGCTGCCGGGCGCGGAGGCAGGGTCTCAACGGATGACTGGCCC
TGTGATTGGGTGGCCCTGGGAGCTTGCGGTTCTGCCTCCAACATTCCGAT
CCCGCCTCCTTGCCTTACTCCGTCCCCGGAGCGGGGATCACCTCAACTTG
ACTGTGGTCCCCCAGCCCTCCAAGAAATGCCCATAAACCAAGGAGGAGAG
GGTAAAAAATAG

In the invention, the term “embryonic stem cells (ESCs)” refers to a kind of cells isolated from an early embryo (prior to the gastrula stage) or a primordial gonad, which have the characteristics of in vitro proliferation, self-renewal and multi-directional differentiation.

As to the term “induced pluripotent stem cells (iPSCs)”, in 2006, Shinya Yamanaka from Kyoto University in Japan was the first to report the study on induced pluripotent stem cells on world-famous academic journal “Cell”. They had four transcription factor genes, i.e. Oct3/4, Sox2, c-Myc and Klf4, cloned into viral vectors, and then were introduced into mouse fibroblasts. It was found that the fibroblasts could be induced to transform into iPS cells, and the iPS cells were similar to embryonic stem cells in terms of morphology, gene and protein expression, epigenetic modification status, cell proliferation ability, embryoid body and teratoma formation ability, differentiation ability, et al. Afterwards, scientists around the world have developed other methods, and somatic cells could be reprogrammed into pluripotent stem cells by using the different gene combination and transfection skills.

The term “hematopoietic stem cells (HSCs)” refers to somatic stem cells in the blood system, which are a heterogeneous population having a long-term self-renewal capacity and a potential of being differentiated into various mature blood cells.

The term “hematopoietic stem/progenitor cells (HSPCs)” refers to somatic stem/progenitor cells in the blood system, which are a heterogeneous population having a long-term self-renewal ability and a potential of being differentiated into various mature blood cells. Hematopoietic stem/progenitor cells include hematopoietic stem cells and hematopoietic progenitor cells.

Preferably, the embryonic stem cells, the induced pluripotent stem cells or the hematopoietic stem/progenitor cells as involved in the invention are derived from mammal cells such as human cells.

The term “nucleic acid construct” as used herein refers to a single-stranded or double-stranded nucleic acid molecule, preferably an artificially constructed nucleic acid molecule. Optionally, the nucleic acid construct further comprises one or more operably linked regulatory sequences.

In the invention, the term “operably linking” refers to a functional steric arrangement of two or more nucleotide regions or nucleic acid sequences. The “operably linking” can be achieved by gene recombination.

In the invention, the term “vector” refers to a nucleic acid carrier tool which can have a polynucleotide inhibiting a certain protein inserted therein. For example, vectors include: plasmid; phagemid; cosmid; artificial chromosome such as yeast artificial chromosome (YAC), bacterial artificial chromosome (BAC) or P1 derived artificial chromosome (PAC); phage such as λ phage or M13 phage and animal virus, etc. Animal viruses as vectors include retroviruses (including lentiviruses), adenoviruses, adeno-associated viruses, herpesviruses (such as herpes simplex virus), poxviruses, baculoviruses, papillomaviruses, papillomaviruses (such as SV40). A vector may contain a variety of elements that control expression.

In the invention, the term “a host cell” refers to a cell into which a vector is introduced, including the following cell types, for example, prokaryotic cells such as Escherichia coli or Bacillus subtilis, fungal cells such as yeast cells or Aspergillus, insect cells such as Drosophila S2 cells or Sf9 cells, or, for example, fibroblasts, CHO cells, COS cells, NSO cells, HeLa cells, BHK cells, HEK 293 cells, or animal cells such as human cells.

The term “an effective amount” refers to a dose that can treat, prevent, alleviate and/or ease the disease or condition of the invention in a subject, when the subject is an individual. When the subject is a cell, it refers to a dose that generates a desired effect or exert an desired action, for example, the dose in a cell is 1 μM-100 μM , 5 μM-50 μM, 5 μM-30 μM, 5 μM-25 μM, 5 μM-20 μM, 5 μM-15 μM, 5 μM-10 μM, 10 μM-25 μM, 10 μM-15 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 50 μM or 100 μM, or 1 μg/mL or 2 μg/mL, etc.

The term “disease and/or condition” refers to a physical status of the subject, which is associated with the disease and/or condition of the invention.

The term “subject” may refer to a patient or an animal that receives the pharmaceutical composition of the present invention to treat, prevent, alleviate and/or ease the disease or condition of invention, particularly a mammal, such as a human, a dog, a monkey, a cow, and a horse.

In the invention, knockdown of DNA or RNA includes, but is not limited to, complete knockout and partial knockout. Complete knockout refers to the reduction of the level of DNA or RNA of interest or the level of the expressed protein to an almost undetectable level (in fact, it is generally difficult to 100% knockout DNA or RNA of interest). Partial knockout means that the degree of knockout is greater than zero, but less than that of complete knockout.

In the invention, unless otherwise specified, the concentration unit μM represents μmol/L, the concentration unit mM represents mmol/L, and the concentration unit nM represents nmol/L.

In the invention, when the added amount of a drug is mentioned, unless otherwise specified, it generally refers to the final concentration of a drug after the addition.

### BENEFICIAL EFFECTS OF THE INVENTION

By downregulating the expression level of NOTCH4 gene, or inhibiting Notch pathway, it can significantly promote the production of hematopoietic stem/progenitor cells and/or megakaryocyte in vitro, which is favorable for improving the yield of megakaryocytes in vitro, and reducing cost. The inventor also found that Notch pathway inhibitors mainly promote the production of hematopoietic stem/progenitor cells, megakaryocyte progenitor cells and mature megakaryocytes. The invention provides new solutions for clinical translation of stem cells, for production of hematopoietic stem/progenitor cells and megakaryocytes in vitro, for clinical transplantation, and for treatment of a disease associated with abnormal platelet.

### DESCRIPTION OF THE DRAWINGS

FIG. 1: cell photos taken under an inverted microscope. Magnification: 4 folds. The sample in FIG. 1A was a normal control (Aicas9); the sample in FIG. 1B was the iPSCs having NOTCH4 partially knocked out (NOTCH4 heter); and the sample in FIG. 1C was the iPSCs having NOTCH4 completely knocked out (NOTCH4 homo). In FIG. 1A-FIG. 1C, the black sphere in middle was embryoid body (EB), and the surrounding single cells were hematopoietic cells.

FIG. 2: a flow cytometric graph, wherein FIG. 2A-FIG. 2C represent the result of the populations of hematopoietic stem/progenitor cells HSPCs (CD34⁺CD45⁺), megakaryocyte progenitor cells MKPs (CD34⁺CD41⁺) and mature megakaryocytes MKs (CD41⁺CD42⁺) of normal control Aicas9, respectively; FIG. 2D-FIG. 2F represent the result of the populations of HSCs, MKPs and MKs of NOTCH4 heter, respectively; FIG. 2G-FIG. 2I represent the result of the populations of HSCs, MKPs and MKs of NOTCH4 homo, respectively.

FIG. 3: a statistical graph of flow cytometry result, wherein the ordinate represents the percent of positive cell populations in the total number of cells. Normal control (Aicas9), iPSCs having NOTCH4 partially knocked out (NOTCH4 heter), and iPSCs having NOTCH4 completely knocked out (NOTCH4 homo), were compared with respect to their differentiation into HSPCs (CD34⁺CD45⁺), MKPs (CD34⁺CD41⁺) and MKs (CD41⁺CD42⁺).

FIG. 4: a statistical graph of flow cytometry result, wherein the ordinate represents the increased or decreased fold of the cell number of HSPCs (CD34⁺CD45⁺), MKPs (CD34⁺CD41⁺) and MKs (CD41⁺CD42⁺), into which iPSCs having NOTCH4 partially knocked out (NOTCH4 heter) and iPSCs having NOTCH4 completely knocked out (NOTCH4 homo) were differentiated, as compared to the control group WT; control group WT, set as 1. N=3, **, p<0.01; ***, p<0.001.

FIG. 5: a statistical graph of flow cytometry result, after the addition of RO4929097 (10 μM) at Day 0, wherein the ordinate represents the percent of positive cell populations in the total number of cells.

FIG. 6: a statistical graph of flow cytometry result, after the addition of different Notch pathway inhibitors at each concentration at different time points to the in vitro megakaryocyte lineage differentiation system of normal iPSC BC1. FIGS. 6A, 6B, and 6C represent the percent of populations of HSPCs (CD34⁺CD45⁺), MKPs (CD34⁺CD41⁺) and MKs (CD41⁺CD42⁺), respectively. *, p<0.05.

FIG. 7: a statistical graph of flow cytometry result, after the addition of the inhibitors RO4929097, L-685458 and DAPT at different time points to the in vitro megakaryocyte lineage differentiation system of normal iPSC BC1, wherein the ordinate represents the increased or decreased fold of different cell populations as compared to the control group after the addition of different inhibitors; the control group, to which DMSO was added, was set as 1. In FIG. 7A, 10 μM R04929097, 10 μM L-685458, and 10 μM DAPT were added at Day 2 after differentiation, respectively; in FIG. 7B, 10 μM RO4929097, 10 μM L-685458, 10 μM DAPT were added at Day 5 after differentiation, respectively. *, p<0.05; **, p<0.01; ***, p<0.001.

FIG. 8: a diagram of the cell number of megakaryocytes (CD41⁺CD42⁺) determined by flow cytometry at Day 14, after the addition of the inhibitor RO4929097 or DAPT at different time points to the in vitro megakaryocyte lineage differentiation system of normal iPSC BC1.

FIG. 9: a statistical graph of flow cytometry result, after the addition of the inhibitor RO4929097 or DAPT to the in vitro megakaryocyte lineage differentiation system of normal iPSC BC1 at Day 2, wherein the ordinate represents the increased or decreased fold of the cell number of different cell populations as compared to the control group after the addition of different inhibitors; the control group, to which DMSO was added, was set as 1. N=3, *, p<0.05; **, p<0.01; ***, p<0.001.

FIG. 10: a statistical graph of flow cytometry result, after the addition of the inhibitor RO4929097 or DAPT to the in vitro megakaryocyte lineage differentiation system of normal iPSC BC1 at Day 5, wherein the ordinate represents the increased or decreased fold of the cell number of different cell populations as compared to the control group after the addition of different inhibitors; the control group, to which DMSO was added, was set as 1. N=3, *, p<0.05; **, p<0.01; ***, p<0.001.

FIG. 11: the ability of megakaryocyte progenitor cells to form mature MK colony (CFU-MK), after the addition of the inhibitor R04929097 or DAPT to the in vitro megakaryocyte lineage differentiation system of normal iPSC BC1 at Day 5. The control group, to which DMSO was added, was set as 1. N=3, *, p<0.05.

FIG. 12: a statistical graph of flow cytometry result after the addition of the inhibitor RO4929097 or DAPT to the in vitro megakaryocyte lineage differentiation system of normal iPSC BC1 at Day 8, wherein the ordinate represents the increased or decreased fold of the cell number of different cell populations as compared to the control group after the addition of different inhibitors; the control group, to which DMSO was added, was set as 1. N=3, *, p<0.05; **, p<0.01.

FIG. 13: a statistical graph of flow cytometry result, after the addition of the inhibitor RO4929097 or DAPT to the in vitro megakaryocyte lineage differentiation system of normal iPSC BC1 at Day 11, wherein the ordinate represents the increased or decreased fold of the cell number of different cell populations as compared to the control group after the addition of different inhibitors; the control group, to which DMSO was added, was set as 1. N=3, ***, p<0.001.

FIG. 14: a statistical graph of flow cytometry result, after the addition of different Notch pathway inhibitors to umbilical cord blood-derived CD34⁺ cells, wherein the ordinate represents the increased fold of different cell populations as compared to the control group after the addition of different inhibitors; the control group, to which DMSO was added, was set as 1.

FIG. 15: a statistical graph of flow cytometry result, after the addition of the Notch pathway inhibitor RO4929097 or DAPT during the in vitro megakaryocyte lineage differentiation of umbilical cord blood-derived CD34⁺ cells, wherein the ordinate represents the increased fold of the cell number of mature megakaryocytes (CD41⁺CD42⁺) as compared to the control group, after the addition of different inhibitors; the control group, to which DMSO was added, was set as 1. N=3, *, p<0.05.

FIG. 16: the ability of megakaryocyte progenitor cells to form mature MK colony (CFU-MK), after the addition of the Notch pathway inhibitor RO4929097 or DAPT during the in vitro megakaryocyte lineage differentiation of umbilical cord blood-derived CD34⁺ cells. The control group, to which DMSO was added, was set as 1. N=3, *, p<0.05; **, p<0.01.

FIG. 17: after the addition of the inhibitor RO4929097 or DAPT to the in vitro megakaryocyte lineage differentiation system of normal iPSC BC1 at Day 5, megakaryocytes were further subjected to induced differentiation and maturation, and the ability of forming polyploids was detected. The ordinate represents the proportion of megakaryocytes having chromosome number>8N in CD61⁺ megakaryocytes, after the addition of different inhibitors or DMSO. N=3, n.s., there was no significant difference among the results.

FIG. 18: after the addition of the Notch pathway inhibitor RO4929097 or DAPT during the in vitro megakaryocyte lineage differentiation of umbilical cord blood-derived CD34⁺ cells, the ability of forming megakaryocyte polyploids was detected. The ordinate represents the proportion of megakaryocytes having chromosome number≥8N in CD61⁺ megakaryocytes, after the addition of different inhibitors or DMSO. N=3, n.s., there was no significant difference among the results.

FIG. 19: after the addition of the inhibitor R04929097 or DAPT to the in vitro megakaryocyte lineage differentiation system of normal iPSC BC1 at Day 5, megakaryocytes were further subjected to induced differentiation and maturation, and the ability of forming proplatelet was detected. FIGS. 19A-C showed the bright-field cell photos taken under an inverted microscope, wherein the sample in FIG. 19A was DMSO control group; the sample in FIG. 19B was the group added RO4929097; and the sample in FIG. 19C was the group added DAPT. In FIG. 19A-FIG. 19C, the arrowhead represents proplatelets . In FIG. 19D, the ordinate represents the proportion of proplatelet-bearing MKs in 100 differentiated cells as counted. N=8, n.s., there was no significant difference among the results.

FIG. 20: after the addition of the Notch pathway inhibitor RO4929097 or DAPT during the in vitro megakaryocyte lineage differentiation of the umbilical cord blood-derived CD34⁺ cells, the ability of forming proplatelet was detected. FIG. 20A-C showed the bright-field cell photos taken under an inverted microscope, wherein the sample in FIG. 20A was DMSO control group; the sample in FIG. 20B was the group added RO4929097; and the sample in FIG. 19C was the group added DAPT. In FIG. 20A-FIG. 20C, the arrowhead represents proplatelets. In FIG. 20D, the ordinate represents the proportion of proplatelet-bearing MKs in 100 differentiated cells as counted. N=6, n.s., there was no significant difference among the results.

### SPECIFIC MODES FOR CARRYING OUT THE INVENTION

The embodiments of the invention are illustrated by reference to the following examples. A person skilled in the art would understand that the following examples are used only for illustrating the invention, but not intended to limit the protection scope of the present invention. In the case where the specific techniques or conditions are not indicated in the Examples, they are carried out according to the techniques or conditions described in the documents in the art (see, for example, Sambrook J et al., Molecular Cloning: A Laboratory Manual (Third Edition), translated by Huang Peitang et al., Science Press) or according to the manuals of products. The reagents or devices, the manufacturers of which are not indicated, are the conventional products that are commercially available.

In the invention, the abbreviations have the following meanings:

bFGF: Basic fibroblast growth factor.

BMP4: Bone morphogenetic protein 4.

SCF: Stem cell factor.

VEGF: Vascular endothelial growth factor.

### EXAMPLE 1

### Obtainment of a Cell System Comprising Hematopoietic Stem/Progenitor Cells, Megakaryocyte Progenitor Cells and Mature Megakaryocytes by in vitro Differentiation of Induced Pluripotent Stem Cells (iPSCs)

1. Experimental Materials and Reagents

Normal human iPSCs cell line BC1, derived from bone marrow CD34+ cells of a healthy adult volunteer, and iPSCs cell line formed by reprogramming through episomal plasmid transfection. As to the particular preparation steps, please see: Dowey S N, Huang X, Chou B K, Ye Z, Cheng L, Generation of integration-free human induced pluripotent stem cells from postnatal blood mononuclear cells by plasmid vector expression, Nat Protoc. 2012 Nov; 7(11):2013-21.

E8 medium, IMDM and Ham's F12: purchased from ThermoFisher SCIENTIFIC.

Antibodies used in flow cytometry: purchased from eBioscience.

2. Experimental Method

BC1 cell line was cultured in E8 medium, under the culture condition of 37° C., 5% CO₂, and was passaged at (1:5)-(1:10) every 3-4 days depending on the concentration of cells. The cells were incubated in 0.5 mM EDTA, at room temperature for 2 min, and then were re-suspended and split up and down with E8 medium, and transferred to a culture dish that had been coated with Vitronectin one hour before.

During differentiation, spin-embryoid body (spin-EB) method was used (Ng E S, Davis R, Stanley E G, Elefanty A G, A protocol describing the use of a recombinant protein-based, animal product-free medium (APEL) for human embryonic stem cell differentiation as spin embryoid bodies, Nat Protoc. 2008; 3(5): 768-76.). At Day 0 (D0) after differentiation, iPSCs reached a confluence of above 85% (confluence refers to the percentage of the surface of a culture dish that is covered by adherent cells). After digestion with Accutase at 37° C. for 3 min, the cells were neutralized and split in SFM medium (50% IMDM, 50% Ham's F12, and the added reagents and factors including: human albumin, monothioglycerol, Glutamax, Chemically Defined Lipid Concentrate, L-Ascorbic acid 2-phosphate sesquimagnesium salt hydrate, ITS-X), counted, and centrifuged. After the cells were re-suspended in SFM, cytokines, i.e. 10 μM ROCK inhibitor (Rho-associated protein kinase inhibitor), 10 ng/ml bFGF, and 10 ng/ml BMP4, were added. Cells were seeded to a round-bottom 96-well plate, at 3000 or 5000 cells/50 μl for each well (3000 or 5000 cells were seeded to each well, in a volume of 50 μl for each well). At Day 2, 50 μl SFM medium+10 ng/ml bFGF+10 ng/ml BMP4+100 ng/ml SCF+20 ng/ml VEGF was added to each well. At Day 5, Day 8, and Day 11, 50 μl SFM medium+10 ng/ml bFGF+10 ng/ml BMP4+50 ng/ml SCF+10 ng/ml VEGF was added to each well, wherein 100 μl old medium was pipetted off at Day 8, and 10 ng/ml thrombopoietin (TPO) was added to each well at Day 11.

At Day 14 (D14), single cells (suspending cells) surrounding embryoid bodies (EBs) were collected. The cells were used in the following flow cytometry.

The cell surface markers (CD34, CD45, CD41, CD42) of the collected cells were detected by flow cytometer. The assay was as followed.

The detection was carried out in four tubes: the first tube was a negative tube that was not labeled with any flow antibody; the second tube was labelled with two antibodies CD34-APC and CD45-PE; the third tube was labeled with CD34-APC and CD41-PE; and the fourth tube was labeled with CD41-PE and CD42-APC, wherein CD34⁺CD45⁺represented the population of hematopoietic stem/progenitor cells (HSPCs), CD34⁺CD41⁺ represented the population of megakaryocyte progenitor cells (MKPs), and CD41⁺CD42⁺ represented the population of mature megakaryocytes (MKs).

The flow cytometry result showed that the collected cells included hematopoietic stem/progenitor cells, megakaryocyte progenitor cells and mature megakaryocytes.

### EXAMPLE 2-1

### Verification of the Role of NOTCH4 Gene in Megakaryocyte Differentiation in vitro by CRISPR/Cas9 Knockout Experiment (1)

1. Experimental Materials

iPSCs cell line Aicas9 (an improved BC1 cell line) capable of inducing the expression of Cas9 protein, wherein doxycycline-inducible Cas9 expression cassette was introduced into BC1 cell line, and the cassette was inserted into the AAVS1 gene locus. As to the particular preparation steps, please see: Gonzalez F, Zhu Z, Shi Z D, Lelli K, Verma N, Li Q V, Huangfu D, An iCRISPR platform for rapid, multiplexable, and inducible genome editing in human pluripotent stein cells, Cell Stem Cell. 2014 Aug. 7; 15(2):215-26.

pLKO lentiviral expression vector, purchased from Addgene.

Plasmid extraction was carried out by using endo-free Plasmid Midi Kit, purchased from Kangwei Biotechnology Co., Ltd.

The electro-transfection reagent was Human Stein Cell Nucleofector® Kit 1 purchased from Lonza Company.

2. Experimental Method

(1) Construction of pLKO-gRNA Expression Vector

pLKO Lentiviral Expression Vector was Used.

Depending on the NOTCH4 gene sequence (SEQ ID NO: 2), X20NGG characteristic sequences were looked for, off-target sequences were excluded by alignment analysis, and the 2 gRNA thus determined were:

NOTCH4-gRNA1:1305-1327: ccctgccagaacgcccagctctg (SEQ ID NO: 3); and

NOTCH4-gRNA2: 2837-2859: cccaccgggcttcgagggccatg (SEQ ID NO: 4).

The gRNA sequences and complementary sequences thereof were separately synthesized, annealed and then ligated to the BfuAI enzyme-cleaved pLKO vector, to construct the pLKO-gRNA expression vectors (NOTCH4-gRNA1-pLKO and NOTCH4-gRNA2-pLKO), which were transformed, and grown onto an agar plate. Clones were picked, and identified by sequencing.

(2) Construction of iPSC cell line having NOTCH4 completely knocked out (NOTCH4 homo) and iPSC cell line having NOTCH4 partially knocked out (NOTCH4 heter).

The NOTCH4-gRNA1-pLKO plasmid and NOTCH4-gRNA2-pLKO plasmid as constructed above were extracted. NOTCH4-gRNA1-pLKO and NOTCH4-gRNA2-pLKO plasmid were simultaneously electro-transformed into Aicas9 cell line, so as to enhance the knockout efficiency. Doxycycline was added one day before electro-transformation, to induce the expression of Cas9 protein. After electro-transformation, the cells were cultured in E8 medium for 3 days, under the culture condition of 37° C., 5% CO₂, and doxycycline was added simultaneously. Flow cytometer was used to sort GFP⁺ single cells to a 96-well plate, and after monoclones grew, the cells were passaged and proliferated. The cells were cryopreserved for cell stock , and meanwhile the cells were lysed for genotype identification.

PCR primers were designed at 5′ end and 3′ end of the NOTCH4-gRNA1 and NOTCH4-gRNA2 cleavage site:

NOTCH4-F:
(SEQ ID NO: 5)
GAAGGAGCCCAGGGTGTTATG;
NOTCH4-R:
(SEQ ID NO: 6)
TAGGAAGAGGGACCAGTGATGT.

After the cells were lysed, the F+R primer pair was used to carry out PCR, and the PCR products were subjected to agarose gel electrophoresis and Sanger Sequencing. If the F+R PCR product had two bands (i.e. 1732 bp band and 199 bp band), the 1732 bp band was identified as WT by Sanger Sequencing, and the 199bp band was identified to have a large fragment deleted at two cleavage sites by Sanger Sequencing, the cell clone was NOTCH4 heterogenous iPSC; if the F+R PCR product only had the 199 bp band, it was homogenous iPSC, and the PCR product was identified by Sanger Sequencing. The iPSC cell line having NOTCH4 completely knocked out and iPSC cell line having NOTCH4 partially knocked out were constructed successfully.

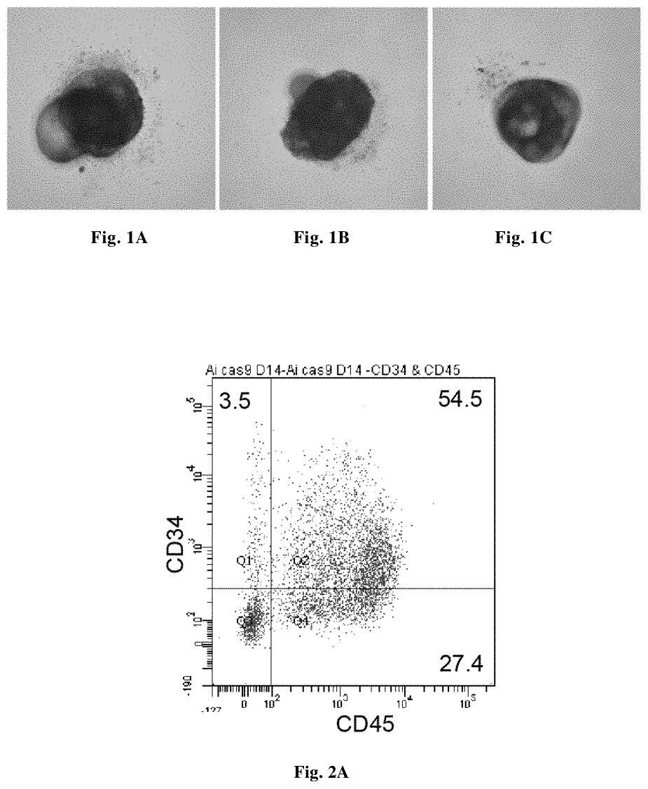

(3) Normal control iPSCs cell line (Aicas9), iPSC cell line having NOTCH4 partially knocked out, and iPSC cell line having NOTCH4 completely knocked out, were compared with respect to the ability of being differentiated into hematopoietic stem/progenitor cells, megakaryocyte progenitor cells and mature megakaryocytes in vitro. As to the steps of in vitro differentiation, please refer to Example 1 above. The results were shown in FIGS. 1A-1C.

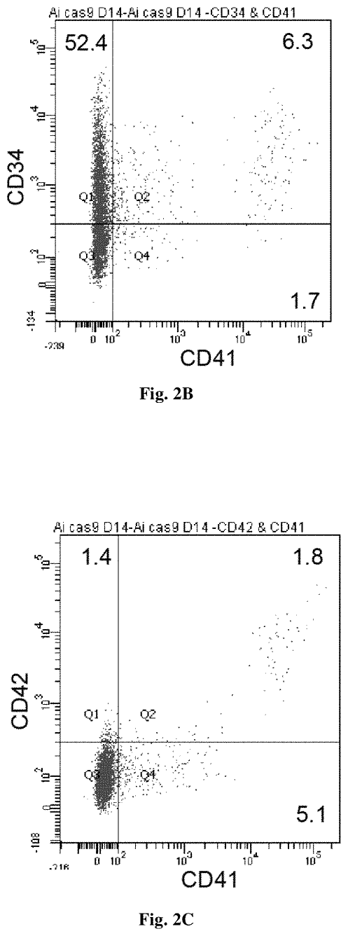

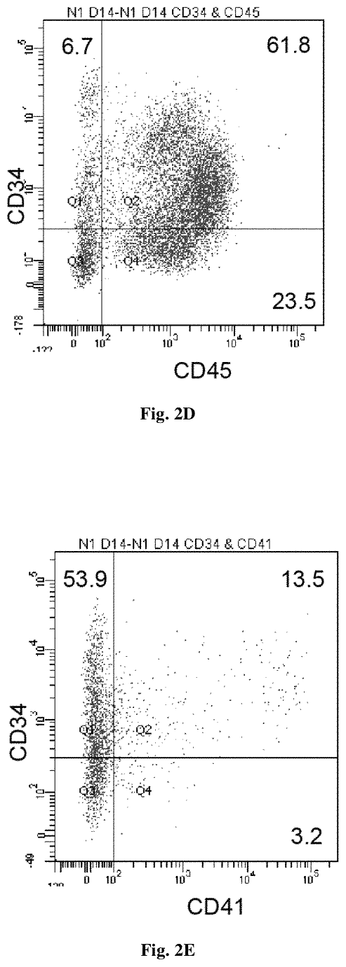

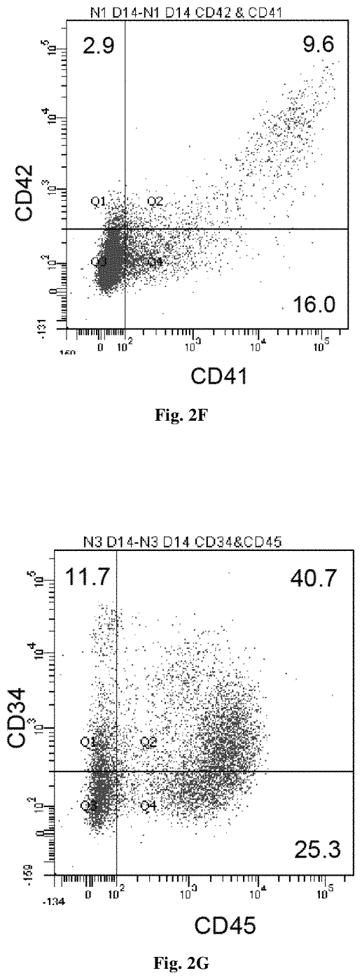

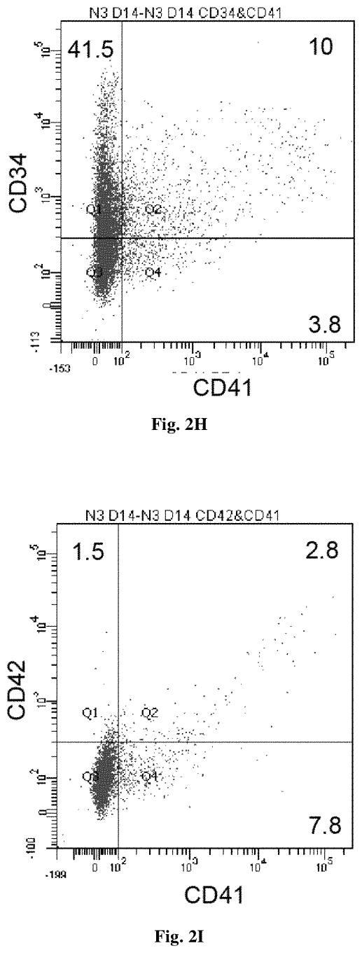

(4) The hematopoietic cells were collected for flow cytometry analysis. As to the steps, please refer to Example 1. The results were shown in FIGS. 2A-2I.

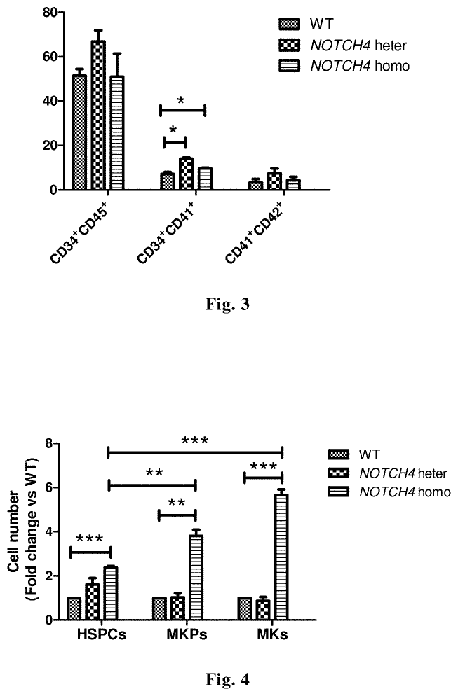

The experiment above was repeated, and the flow cytometry results obtained were subjected to statistics. The result was shown in FIG. 3.

### 3. Experimental Result

As shown in FIGS. 1A-1C, the black sphere in middle was embryoid body (EB), and the surrounding single cells were hematopoietic cells.

The flow cytometry results were shown in FIGS. 2A-2I. Moreover, the statistical result in FIG. 3 showed: the cell line having NOTCH4 partially knocked out (NOTCH4 heter) and the cell line having NOTCH4 completely knocked out (NOTCH4 homo) were superior to the control group with respect to the ability of producing megakaryocyte progenitor cells in vitro. After partially knocking out NOTCH4, the proportion of the megakaryocyte progenitor cells produced was increased by 95% as compared to the control group (*, p<0.05), while after completely knocking out NOTCH4, the proportion of the megakaryocyte progenitor cells produced was increased by 35% (*, p<0.05).

The result above showed that NOTCH4 gene had an effect on inhibiting human megakaryocyte differentiation in vitro. After partially knocking out NOTCH4 and completely knocking out NOTCH4, the hematopoietic stem/progenitor cells and mature megakaryocytes produced were increased as compared to the control group.

### EXAMPLE 2-2

### Verification of the Role of NOTCH4 Gene in Megakaryocyte Differentiation in vitro by CRISPR/Cas9 Knockout Experiment (2)

1. Experimental Materials

The materials were the same as those in Example 2-1.

2. Experimental Method

(1) Construction of pLKO-gRNA Expression Vector

pLKO Lentiviral Expression Vector was Used.

Only the NOTCH4-gRNA1-pLKO expression vector was used in electro-transfection, wherein NOTCH4-gRNAl: 1305-1327: ccctgccagaacgcccagctctg (SEQ ID NO: 3). The inventor found in the experiment above that the knockout efficiency of gRNA was too high, and therefore only one gRNA was used.

The steps for constructing the vector was the same as the corresponding steps in Example 2-1.

(2) Construction of iPSC cell line having NOTCH4 completely knocked out (NOTCH4 homo) and iPSC cell line having NOTCH4 partially knocked out (NOTCH4 heter)

The steps for constructing a monoclonal cell line was the same as the corresponding steps in Example 2-1.

PCR primers were designed at 5′ end and 3′ end of NOTCH4-gRNA3 cleavage site:

NOTCH4-F:
(SEQ ID NO: 5)
GAAGGAGCCCAGGGTGTTATG;
NOTCH4-R:
(SEQ ID NO: 7)
GCTAGAAACGGCTCCCTCTG.

The monoclonal cell line was subjected to genotype identification: after the cells were lysed, the F+R primers were used to carry out PCR, and the PCR products were subjected to agarose gel electrophoresis and Sanger Sequencing. The result showed that the inventor had successfully constructed the iPSC cell line having NOTCH4 completely knocked out and the iPSC cell line having NOTCH4 partially knocked out.

(3) Normal control WT cell line (without gene knockout), the iPSC cell line having NOTCH4 partially knocked out, and the iPSC cell line having NOTCH4 completely knocked out NOTCH4 were compared with respect to the ability of being differentiated into hematopoietic stem/progenitor cells, megakaryocyte progenitor cells and mature megakaryocytes in vitro. As to the operation of in vitro differentiation and the collection of hematopoietic cells for flow cytometry, please refer to the Example 1 above.

The experiment above was repeated for at least three times, and the flow cytometry results obtained were subjected to statistics. The result was shown in FIG. 4.

3. Experimental Result

The statistical result in FIG. 4 showed: the iPSC cell line having NOTCH4 completely knocked out (NOTCH4 homo) was significantly superior to the control group WT with respect to the ability of producing hematopoietic stem/progenitor cells, megakaryocyte progenitor cells and megakaryocyte in vitro. After completely knocking out NOTCH4, the number of hematopoietic stem/progenitor cells produced was increased by 2.4 folds as compared to the control group WT, and the number of megakaryocyte progenitor cells produced was increased by 3.8 folds, and the number of megakaryocytes produced was increased by 5.7 folds. (**, p<0.01; ***, p<0.001)

The results above showed that NOTCH4 gene had the effect on inhibiting human hematopoietic and megakaryocytic differentiation in vitro. As compared to the control group, the yield of hematopoietic stem/progenitor cells, megakaryocyte progenitor cells and mature megakaryocytes produced after completely knocking out NOTCH4, was significantly increased.

### EXAMPLE 3-1

### Notch Pathway Inhibitors Could Promote the Production of Megakaryocytes From Stem Cells in vitro

1. Experimental Material

Notch signaling pathway inhibitors: RO4929097, LY411575, PF-03084014, YO-01027, L-685458, DAPT and FLI-06, all of which were purchased from Selleck Company. Among them, FLI-06 mainly inhibited Notch transportation and processing; RO4929097, LY411575, PF-03084014, YO-01027, L-685458, and DAPT were γ-secretase inhibitors. The structural formulae were as followed:

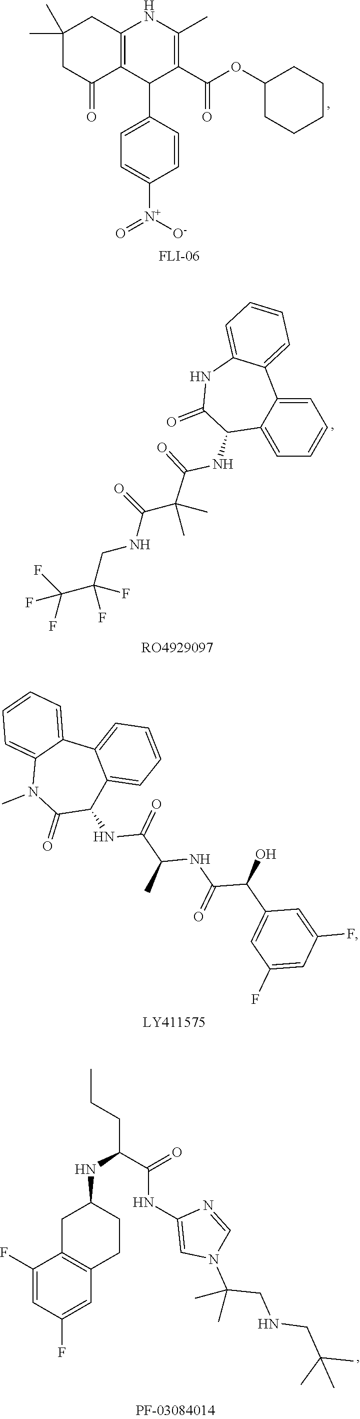 [SMILES: CC1=C(C(=O)OC2CCCCC2)C(c2ccc([N+](=O)[O-])cc2)C2=C(CC(C)(C)CC2=O)N1 ; CC(C)(C(=O)NCC(F)(F)C(F)(F)F)C(=O)N[C@@H]1C(=O)Nc2ccccc2-c2ccccc21 ; C[C@H](NC(=O)[C@@H](O)c1cc(F)cc(F)c1)C(=O)N[C@@H]1C(=O)N(C)c2ccccc2-c2ccccc21 ; CCC[C@H](N[C@H]1CCc2cc(F)cc(F)c2C1)C(=O)Nc1cn(C(C)(C)CNCC(C)(C)C)cn1]

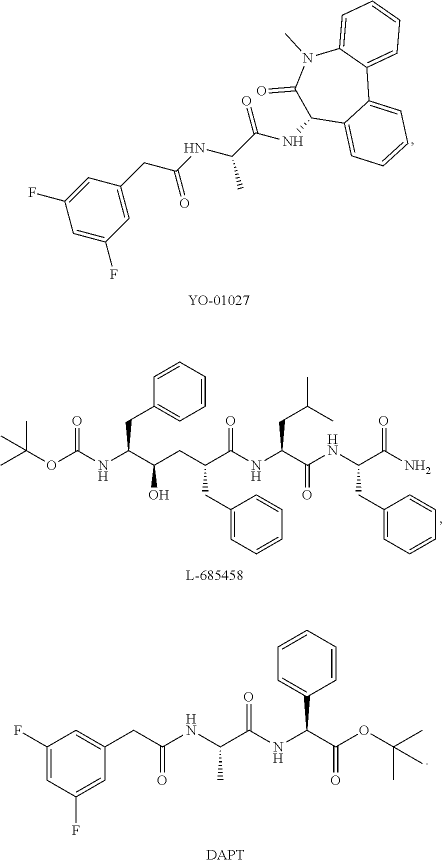 [SMILES: C[C@H](NC(=O)Cc1cc(F)cc(F)c1)C(=O)N[C@@H]1C(=O)N(C)c2ccccc2-c2ccccc21 ; CC(C)C[C@H](NC(=O)[C@H](Cc1ccccc1)C[C@@H](O)[C@H](Cc1ccccc1)NC(=O)OC(C)(C)C)C(=O)N[C@@H](Cc1ccccc1)C(N)=O ; C[C@H](NC(=O)Cc1cc(F)cc(F)c1)C(=O)N[C@H](C(=O)OC(C)(C)C)c1ccccc1]

NOTCH 4 Antibody (E-12) was purchased from Santa-cruz Company.

2. Experimental Method

(1) The BC1 cell line used was the same as the one used in Example 1, and was divided into three groups, wherein two 96-well plates were used for each group, and the cells were seeded at 5000 cells/50 μl to each well. In vitro differentiation was carried out by reference to the steps in Example 1, wherein the step of adding a drug was carried out in accordance with the following steps in I-III groups:

I. At Day 0 after differentiation, 10 μM RO4929097 was added, and DMSO was added to the control well.

II. At Day 2 after differentiation, 10 μM or 15 μM RO4929097, 10 μM or 15 μM LY411575, 5 μM or 10 μM L-685458, 5 μM or 10 μM PF-03084014, 10 μM YO-01027, 5 μM or 10 μM FLI-06, and 1 μg/mL or 2 μg/mL NOTCH4 antibody were added, respectively.

III. At Day 2 after differentiation, 10 μM RO4929097, 10 μM L-685458, and 10 μM DAPT were added, respectively; in addition, at Day 5 after differentiation, 10 μM RO4929097, 10 μM L-685458, and 10 μM DAPT were added, respectively.

The results were compared and observed.

(2) The cells were collected for flow cytometry analysis, and as to the steps, please refer to the Example 1 above.

3. Experimental Result

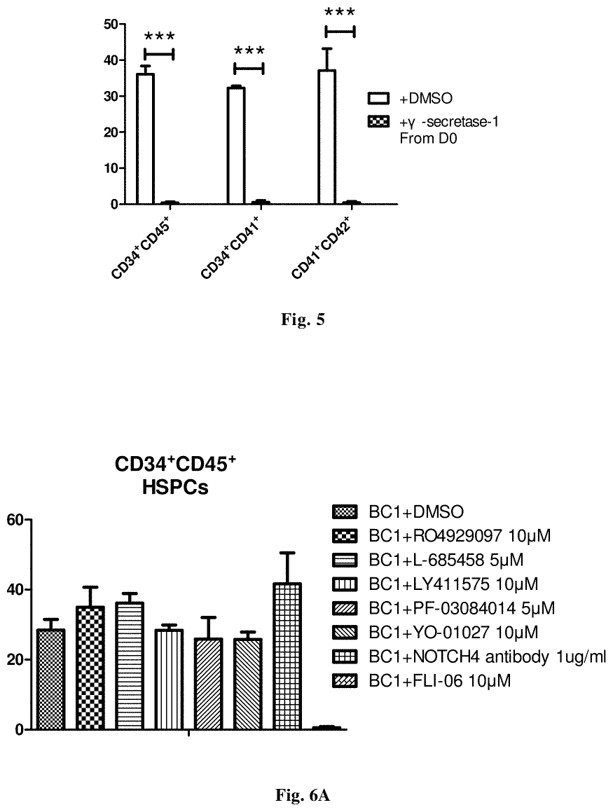

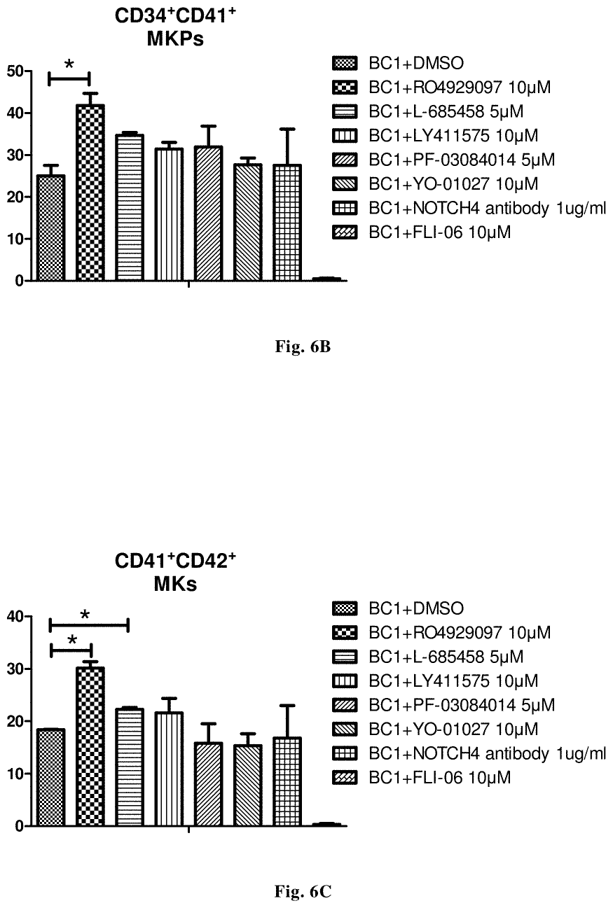

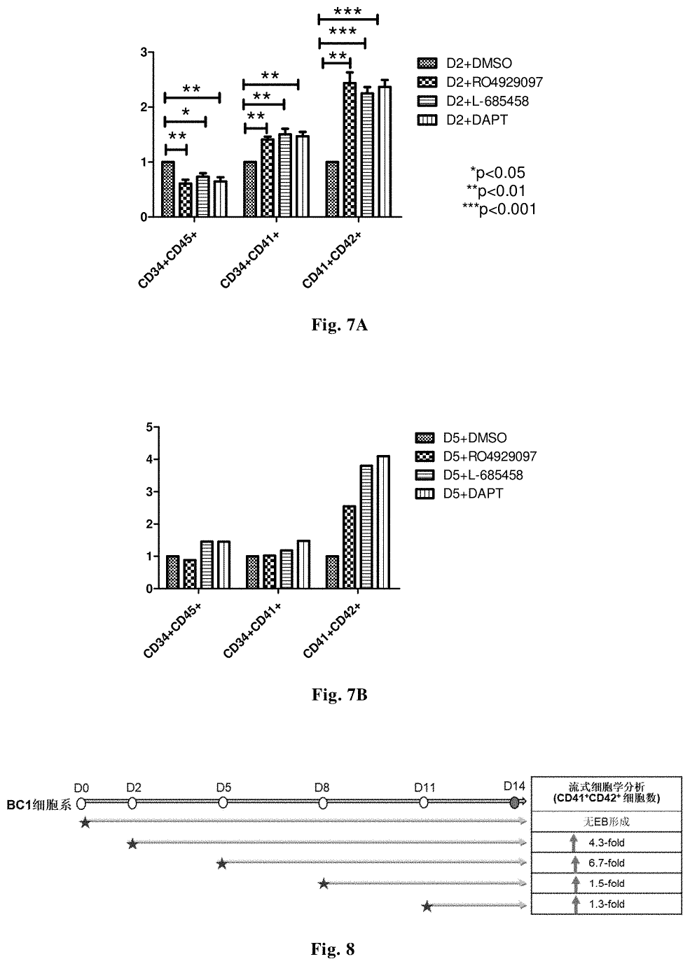

The results were as shown in FIG. 5, FIGS. 6A-6C and FIGS. 7A-7B, respectively.

(1) As shown in FIG. 5, after adding RO4929097 (10 μM) at Day 0, no hematopoietic embryoid body EB was formed; and the proportions of HSPCs (CD34⁺CD45⁺), MKPs (CD34⁺CD41⁺) and MKs (CD41⁺CD42⁺) were close to 0.

(2) As shown in FIGS. 6A-6C, as compared to DMSO control group, after the addition of RO4929097 (10 μM) at Day 2, the proportion of megakaryocyte progenitor cells MKPs (CD34⁺CD41⁺) produced was increased by 67%, and the proportion of mature megakaryocytes MKs (CD41⁺CD42⁺) produced was increased by 64% (p<0.05). After the addition of the inhibitor L-685458 (5 μM) at Day 2, the proportion of mature megakaryocytes produced was also increased by 21% (p<0.05). 15 μM RO4929097 also had a certain effect on promoting the production of MKPs and MKs, but the effect was not as significant as that of 10 μM RO4929097.

(3) As shown in FIG. 7A, after the addition of RO4929097 (10 μM) at Day 2, the proportion of hematopoietic stem/progenitor cells HSPCs (CD34⁺CD45⁺) was decreased by 40%, but the proportion of megakaryocyte progenitor cells MKPs (CD34⁺CD41⁺) produced was increased by about 1.4 folds, and the proportion of mature megakaryocytes MKs (CD41⁺CD42⁺) produced was increased by about 2.5 folds (p<0.01); after the addition of L-685458 (10 μM) at Day 2, the proportion of HSPCs (p<0.05) was decreased by 27% (p<0.05), but the proportion of MKPs produced was increased by about 1.5 folds (p<0.01), and the proportion of matured megakaryocytes MKs produced was increased by about 2.3 folds (p<0.001); after the addition of DAPT (10 μM) at Day 2, the proportion of HSPCs was decreased by 35% (p<0.01), but the proportion of MKPs produced was increased by about 1.5 folds (p<0.01), and the proportion of mature megakaryocytes MKs produced was increased by about 2.4 folds (p<0.001). Moreover, after the addition of inhibitors, the total number of hematopoietic cells produced were increased by nearly 2 folds as compared to the control group.

As shown in FIG. 7B, the addition of an inhibitor at Day 5 had a better effect than the addition of an inhibitor at Day 2. After the addition of RO4929097 (10 μM) at Day 5, the total number of hematopoietic cells did not change, the proportion of HSPCs was decreased by 11%, and the proportion of mature megakaryocytes MKs produced was increased by about 2.6 folds; after the addition of L-685458 (10 μM) at Day 5, the total number of hematopoietic cells formed was increased by 2.3 folds as compared to the control group, the proportion of HSPCs produced was increased by about 1.5 folds, the proportion of MKPs produced was increased by about 1.2 folds, and the proportion of mature megakaryocytes MKs produced was increased by about 3.8 folds; after the addition of DAPT (10 μM) at Day 5, the total number of hematopoietic cells was increased by 2.8 folds as compared to the control group, the proportion of HSPCs produced was increased by about 1.5 folds, the proportion of MKPs produced was increased by about 1.5 folds, and the proportion of mature megakaryocytes MKs produced was increased by about 4.1 folds.

(4) It was found by comparison that 10 μM DAPT worked at Day 5 after in vitro differentiation, had the best effect on promoting the production of megakaryocytes, could promote the production of megakaryocyte progenitor cells by 4 folds, and could promote the production of mature megakaryocytes by nearly 12 folds.

### EXAMPLE 3-2

### Notch Pathway Inhibitors Could Promote the Production of Hematopoietic Stem/Progenitor Cells and Megakaryocytes From Stem Cells in vitro

1. Experimental Materials

Notch pathway inhibitors: RO4929097 and DAPT, both of which were purchased from Selleck Company.

Collagen-based MegaCult-C Kit, purchased from STEMCELL Technologies Company.

2. Experimental Method

(1) The BC1 cell line used was the same as the one used in Example 1, and was divided into three groups, wherein two 96-well plates were used for each group, and the cells were seeded at 3000 cells/50 μl to each well. In vitro differentiation was carried out by reference to the steps in Example 1, wherein the step of adding a drug was carried out in accordance with the following steps in I-V groups:

I. At Day 0 after differentiation, 10 μM RO4929097, and 10 μM DAPT were added, respectively.

II. At Day 2 after differentiation, 10 μM RO4929097, and 10 μM DAPT were added, respectively.

III. At Day 5 after differentiation, 10 μM RO4929097, and 10 μM DAPT were added, respectively.

IV. At Day 8 after differentiation, 10 μM RO4929097, and 10 μM DAPT were added, respectively.

V. At Day 11 after differentiation, 10 μM RO4929097, and 10 μM DAPT were added, respectively.

The results were compared and observed.

(2) The cells were collected for flow cytometry analysis, and as to the steps, please refer to the Example 1 above.

(3) Collection of iPS C-derived CD34⁺ cells: at Day 14, the single cells surrounding embryoid bodies were collected, and CD34⁺ cells were obtained by magnetic bead sorting using CD34 MicroBead Kit.

(4) Megakaryocyte colony forming unit (MK-CFU) experiment: 10,000 CD34⁺ single cells were seeded to collagen-based MegaCult-C Kit. After incubation for 10-12 days, the cells were stained with anti-CD41 antibody and counted. CD41⁺ megakaryocyte colonies were counted.

3. Experimental Result

The results were shown in FIGS. 8-13, respectively. (*, p<0.05; **, p<0.01; ***, p<0.001)

(1) As shown in FIG. 8, after the addition of RO4929097 (10 μM) at Day 0, no hematopoietic embryoid body (EB) could be formed.

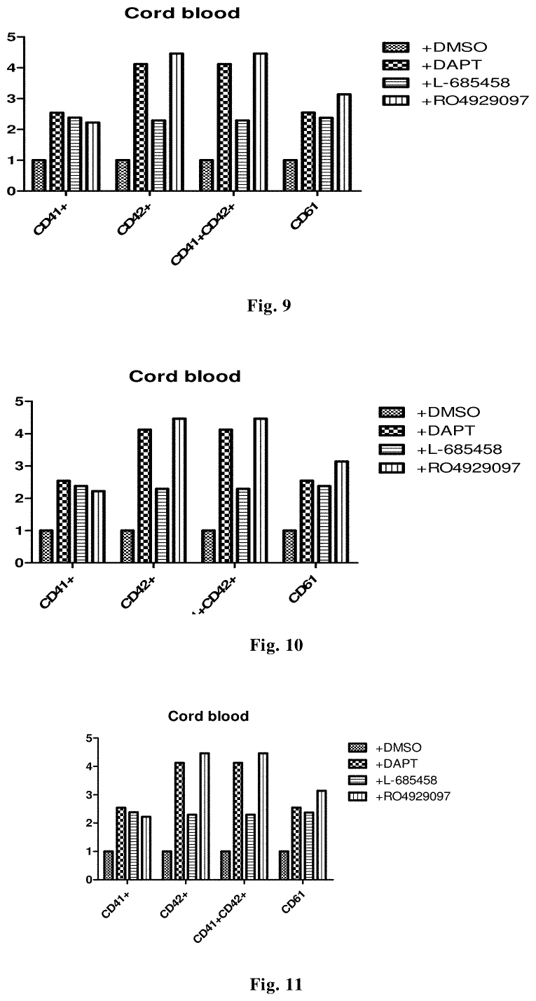

(2) As shown in the statistical result in FIG. 9, after the addition of RO4929097 (10 μM) or DAPT (10 μM) at Day 2, the number of megakaryocyte progenitor cells MKPs (CD34⁺CD41⁺) produced could be increased by 2.5 folds, and the number of mature megakaryocytes MKs (CD41⁺CD42⁺) produced could be increased by 4.3 folds, as compared to DMSO control group.

(3) As shown in the statistical result in FIG. 10-11, after the addition of RO4929097 (10 μM) or DAPT (10 μM) at Day 5, as compared to DMSO control group, not only could the number of hematopoietic stem/progenitor cellsHSPCs (CD34⁺CD45⁺) be increased by 2.8 folds, but also the number of megakaryocyte progenitor cells MKPs (CD34⁺CD41⁺) produced could be increased by about 6.5 folds, and the number of mature megakaryocytes MKs (CD41⁺CD42⁺) produced could be increased by about 6.7 folds. As compared to DMSO control group, after the addition of a Notch pathway inhibitor at Day 5, the ability of megakaryocyte progenitor cells to form mature MK colony (CFU-MK) was increased by 2.2 folds.

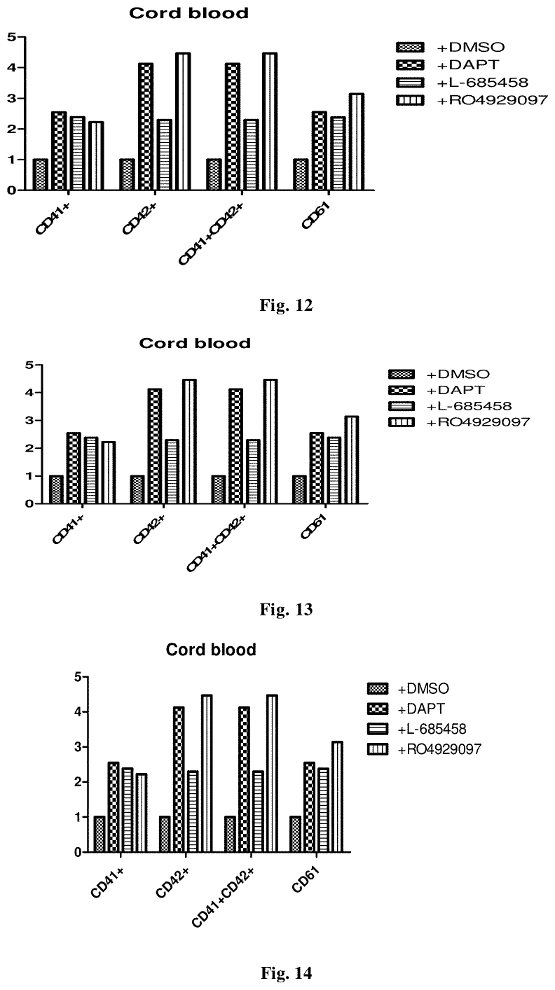

(4) As shown in the statistical result in FIG. 12, after the addition of RO4929097 (10 μM) or DAPT (10 μM) at Day 8, the number of megakaryocyte progenitor cells MKPs (CD34⁺CD41⁺) produced could be increased by 1.5 folds, and the number of mature megakaryocytes MKs (CD41⁺CD42⁺) produced could be increased by 1.5 folds, as compared to DMSO control group.

(5) As shown in the statistical result in FIG. 13, after the addition of RO4929097 (10 μM) or DAPT (10 μM) at Day 11, the number of mature megakaryocytes MKs (CD41⁺CD42⁺) produced could only be increased by 1.3 folds, as compared to DMSO control group.

(6) it could be found by comparison that the inhibitor worked at Day 5 after in vitro differentiation, could not only significantly enhance the production of hematopoietic stem/progenitor cells by about 2.8 folds, but also had the best effect on promoting the production of megakaryocytes, could enhance the production of megakaryocyte progenitor cells by 6.5 folds, and enhance the production of mature megakaryocytes by nearly 6.7 folds.

### EXAMPLE 4-1

### Notch Pathway Inhibitors Could Promote the Production of Megakaryocytes from CD34⁺ in vitro (1)

1. Experimental Materials

Umbilical cord blood was obtained from normal and healthy full-term newborn infants, and the parents of the newborn infants gave their consent.

Ficoll-Paque PLUS was purchased from GE Healthcare Life Sciences Company. CD34 MicroBead Kit was purchased from Miltenyi Biotec Company.

StemSpan™ SFEM II was purchased from STEMCELL Technologies Company.

2. Experimental Method

(1) CD34⁺ cells in umbilical cord blood were obtained by Ficoll-Paque PLUS density gradient centrifugation and magnetic bead sorting using CD34 MicroBead Kit. As to the particular steps, please refer to (GE Healthcare Life Sciences, http://www.gelifesciences.com), (Miltenyi Biotec, http://www.miltenyibiotec.com.cn).

(2) Differentiation of umbilical cord blood-derived CD34⁺ cells into megakaryocytes: umbilical cord blood-derived CD34⁺ cells were counted, and seeded at 1*10⁶ cells to 4 ml StemSpan™ SFEM 11+50 ng/ml SCF+50 ng/ml TPO+50 ng/ml IL-6+20 ng/ml IL-3 in a 12-well plate, with 1 ml medium for each well, and four wells in total, to which 10 μM R04929097, 10 μM L-685458, 10 μM DAPT, and the same volume of DMSO as control were added, respectively, and the time was recorded as Day 0. The liquid was changed every 3 days, and the culture condition was 37° C., 5% CO₂. After incubation for 6 days, the cell density was high, and the cells in each well were divided into two wells of a 12-well plate, with 2 ml medium for each well. The inhibitors were added at the same concnetrations as described above. The cells were collected at D14, counted, and detected for cell surface markers (CD41, CD42, CD61) by flow cytometer.

3. Experimental Result

As shown in FIG. 14:

(1) during the differentiation of umbilical cord blood-derived CD34⁺ cells into mature megakaryocytes (CD41⁺CD42⁺), as compared to control group, the Notch pathway inhibitor DAPT increased the proportion of mature megakaryocytes MK produced by 4.1 folds, RO4929097 increased the proportion of mature megakaryocytes MK produced by 4.5 folds, and L-685458 increased the proportion of mature megakaryocytes MK produced by 2.3 folds; and

(2) as compared with the control group, the Notch pathway inhibitor DAPT increased the proportion of mature megakaryocyte (CD61⁺) by 2.5 folds, RO4929097 increased the proportion of mature megakaryocyte (CD61⁺) by 3.1 folds, and L-685458 increased the proportion of mature megakaryocyte (CD61⁺) by 2.4 folds. CD61 was integrin β3, a cell surface protein, and was involved in the cell adhesion and signal transduction. The proportion of CD61⁺ cells represented the ratio of mature megakaryocytes and platelets.

### EXAMPLE 4-2

### Notch Pathway Inhibitor Could Promote the Production of Megakaryocytes From CD34⁺ Cells in vitro (2)

1. Experimental Materials

The materials were the same as those in Example 4-1.

2. Experimental Method

As to the collection of umbilical cord blood-derived CD34⁺ cells, in vitro megakaryocytic differentiation and detection, please refer to Example 4-1.

Megakaryocyte colony forming unit (MK-CFU) experiment: at Day 14, the cells resulted from induced differentiation were collected, and 7,500 single cells were seeded to collagen-based MegaCult-C Kit. After incubation for 10-12 days, the cells were stained with anti-CD41 antibody and counted. CD41⁺ megakaryocyte colonies were counted.

3. Experimental Result

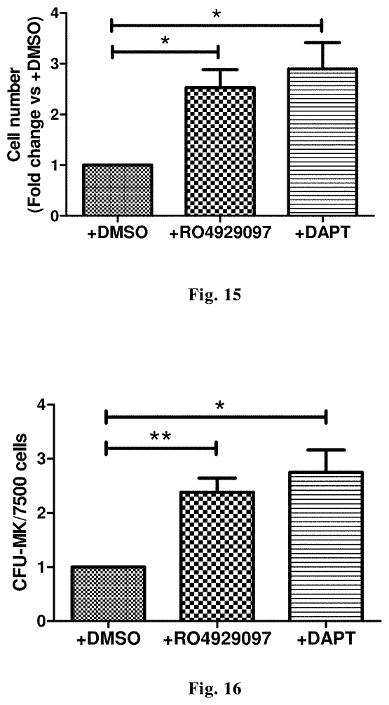

As shown in the statistical result in FIG. 15-16:

(1) During the differentiation of umbilical cord blood-derived CD34⁺ cells into mature megakaryocytes (CD41⁺CD42⁺), as compared to the control group, the Notch pathway inhibitor (10 μM RO4929097 or 10 μM DAPT) increased the number of mature megakaryocytes produced by 2.9 folds; and increased the ability of megakaryocyte progenitor cells to form mature MK colony (CFU-MK) by 2.8 folds.

### EXAMPLE 5

### Megakaryocytes, Which Was Induced by Notch Pathway Inhibitors, had Normal Functions and Characteristics

The polyploid degree of megakaryocytes and the proportion of the proplatelet formed, indicated whether the function and characteristics of mature megakaryocytes were normal or not.

1. Experimental Materials

The materials were the same as those in Example 4-1. Propidium Iodide was purchased from Sigma-Aldrich.

2. Experimental Method

(1) Megakaryocytes were further subjected to induced differentiation and maturation:

iPSC-derived CD34⁺ cells (prepared by the method as described in Example 3-2) and umbilical cord blood-derived CD34⁺ cells were seeded at 1*10⁶ cells per well to 4 ml StemSpan™ SFEM 11+50 ng/ml SCF+50 ng/ml TPO+50 ng/ml IL-6+20 ng/ml IL-3, in a 12-well plate, with 1 ml medium for each well, and 3 wells in total for each cell line, to which 10 μM RO4929097, 10 μM DAPT, and the same volume of DMSO as control, were added, respectively, and the time was recorded as Day 0. The liquid was changed every 3 days, and the culture condition was 37° C., 5% CO₂. After iPSC-derived CD34⁺ cells were incubated for 6 days, and umbilical cord blood-derived CD34⁺ cells were incubated for 9 days, the following experiment was carried out.

(2) Polyploid Detection

Cells were labeled with CD61-FITC flow cytometric antibody at 4° C. for 30 min. The cells were washed with PBS once, and fixed with 70% ethanol at 4° C. overnight. At the second day, the cells were washed with PBS once. After centriguation, 1 ml PBE (PBS containing 2% serum) +100 μg/ml RNase A +50 μg/ml Propidium Iodide, was added, and incubation was carried out in dark at room temperature for 30 min. The cells were washed with PBS once, and the number of cells (≥8N) in CD61⁺ cell population was determined by flow cytometry.

(3) Experiment on Proplatelet Formation

The cells were observed in the bright-field condition under a phase contrast microscope, and the number of proplatelet-bearing MKs in 100 differentiated cells was counted. Proplatelet formation of MKs referred to mature megakaryocytes, which extended long and branched filaments and cytoplasmic protrusions.

3. Experimental Result

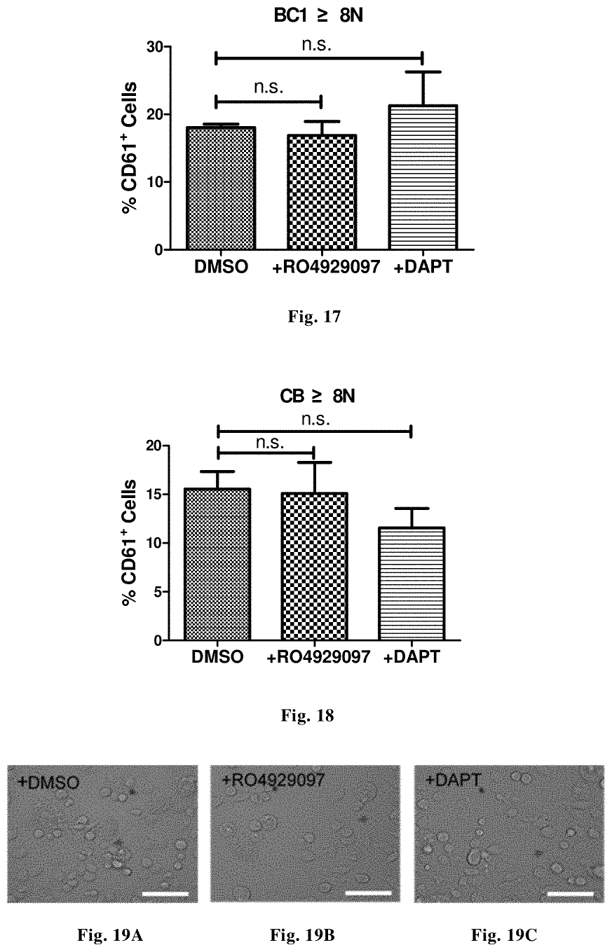

(1) As shown in the statistical result in FIGS. 17-18: during the induced production of mature megakaryocytes from iPSCs-derived CD34⁺ cells (the Notch inhibitor was added since Day 5) and umbilical cord blood-derived CD34⁺ cells, after the addition of the Notch pathway inhibitor (10 μM RO4929097 or 10 μM DAPT), they were similar in terms of the proportion of polyploid megakaryocytes (≥8N) produced, as compared to the DMSO control group:

for iPSCs-derived CD34⁺ cells, the proportion was about 17% or 21% after the addition of RO4929097 or DAPT, while the proportion was about 18% after the addition of DMSO;

for umbilical cord blood-derived CD34⁺ cells, the proportion was about 15% or 12% after the addition of RO4929097 or DAPT; while the proportion was about 16% after the addition of DMSO.

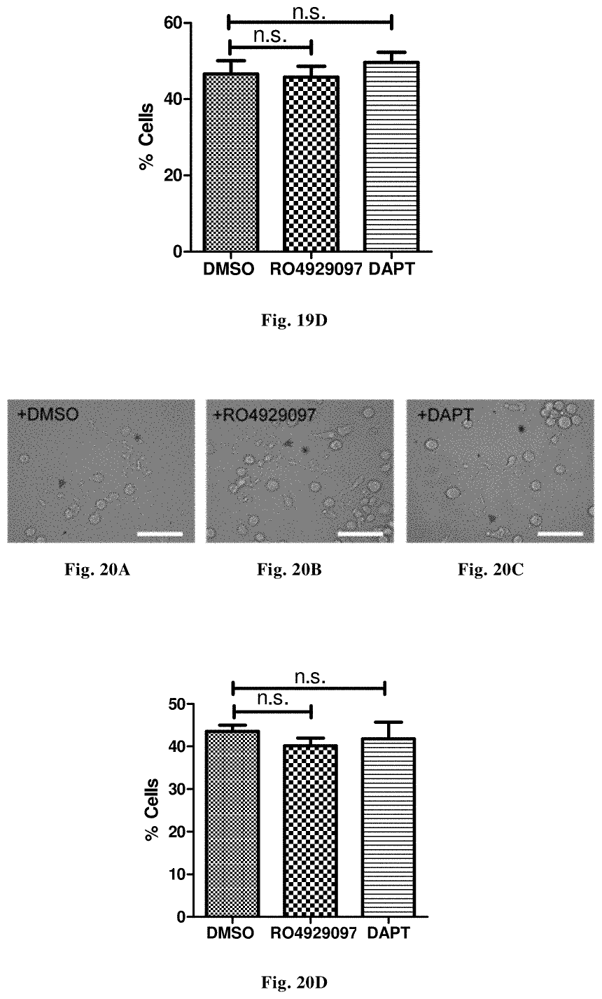

(2) As shown in the statistical results in FIGS. 19A-19D and FIGS. 20A-20D: during the induced production of mature megakaryocytes from iPS Cs-derived CD34⁺ cells and umbilical cord blood-derived CD34⁺ cells, after the addition of a Notch pathway inhibitor (10 μM RO4929097 or 10 μM DAPT), they were also similar in terms of the proportion of the proplatelet-bearing MKs, as compared to the DMSO control group:

for iPSCs-derived CD34⁺ cells, the proportion was about 46% or 50% after the additin of RO4929097 or DAPT, while the proportion was about 47% after the addition of DMSO;

for umbilical cord blood-derived CD34⁺ cells, the proportion was about 40% or 42% after the addition of RO4929097 or DAPT; while the proportion was about 44% after the addition of DMSO.

The results above showed that Notch inhibitors promoted the number of megakaryocytes produced in vitro, and during the maturation of megakaryocytes, as compared to the mature megakaryocytes formed without the addition of inhibitors (control), the mature megakaryocytes formed after the addition of Notch inhibitors also had normal functions and characteristics.

Although the embodiments of the invention have been described in detail, a person skilled in the art would understand that a variety of modifications and replacements may be performed to the details according to all the teachings disclosed therein. These changes all fall into the protection scope of the invention. The scope of the invention is defined by the attached claims and any equivalent thereof.

1-6. (canceled)

7. A recombinant vector, comprising a polynucleotide for completely or partially knocking out NOTCH4 gene.

8. A host cell, comprising the recombinant vector according to claim 7, or in which NOTCH4 gene is completely or partially knocked out.

9. A composition comprising:
the host cell according to claim 8, and cell culture medium.

10. A kit, comprising individually packaged embryonic stem cells, induced pluripotent stem cells or hematopoietic stem/progenitor cells, and a drug or an agent selected from the group consisting of the following items (1) to (4):
(1) a drug for inhibiting or reducing the expression of NOTCH4 gene;
(2) a drug for inhibiting or blocking the activity of NOTCH4 protein;
(3) a drug for completely or partially knocking out NOTCH4 gene; and
(4) a Notch pathway inhibitor.

11. The kit according to claim 30, wherein the γ-secretase inhibitor is one or more selected from the group consisting of RO4929097, L-685458, LY411575, PF-03084014, YO-01027, DAPT and FLI-06.

12. The kit according to claim 10, wherein the drug for inhibiting or blocking the activity of NOTCH4 protein is an antibody against NOTCH4 protein.

13. The kit according to claim 10, wherein the drug for completely or partially knocking out NOTCH4 gene is a polynucleotide for completely or partially knocking out NOTCH4 gene.

14. A method for producing hematopoietic stem/progenitor cells and/or megakaryocytes and/or megakaryocyte progenitor cells in vitro, comprising:
the step of inhibiting or reducing the expression of NOTCH4 gene in an embryonic stem cell, an induced pluripotent stem cell or a hematopoietic stem/progenitor cell; or the step of inhibiting or blocking the activity of NOTCH4 protein in an embryonic stem cell, an induced pluripotent stem cell or a hematopoietic stem/progenitor cell.

15. A method for producing platelet in vitro, comprising:
the step of inhibiting or reducing the expression of NOTCH4 gene in an embryonic stem cell, an induced pluripotent stem cell or a hematopoietic stem/progenitor cell; or
the step of inhibiting or blocking the activity of NOTCH4 protein in an embryonic stem cell, an induced pluripotent stem cell or a hematopoietic stem/progenitor cell.

16. A method for screening a medicament for modulating the production of hematopoietic stem/progenitor cells and/or megakaryocytes and/or megakaryocyte progenitor cells in a mammal, a medicament for treating megakaryocyte dysplasia, or a medicament for modulating platelet production, comprising:
the step of detecting a test medicament for its inhibition or reduction of the expression level of NOTCH4 gene in an embryonic stem cell, an induced pluripotent stem cell or a hematopoietic stem/progenitor cell; or
the step of detecting a test medicament for its inhibition or blockage of the activity level of NOTCH4 protein in an embryonic stem cell, an induced pluripotent stem cell or a hematopoietic stem/progenitor cell.

17-22. (canceled)

23. A method for treating and/or preventing megakaryocyte dysplasia or for treating and/or preventing a disease associated with abnormal platelet, comprising the step of administering to a subject in need thereof an effective amount of the composition according to claim 9, or comprising the step of administering to a subject in need thereof an effective amount of any one of the following items (1) to (4):
(1) a drug for inhibiting or reducing the expression of NOTCH4 gene;
(2) a drug for inhibiting or blocking the activity of NOTCH4 protein;
(3) a drug for completely or partially knocking out NOTCH4 gene; and
(4) a Notch pathway inhibitor.

24. The method according to claim 38, wherein the γ-secretase inhibitor is one or more selected from the group consisting of RO4929097, L-685458, LY411575, PF-03084014, YO-01027, DAPT and FLI-06.

25. The method according to claim 23, wherein the drug for inhibiting or blocking the activity of NOTCH4 protein is an antibody against NOTCH4 protein.

26. The method according to claim 23, wherein the drug for completely or partially knocking out NOTCH4 gene is a polynucleotide for completely or partially knocking out NOTCH4 gene.

27. The recombinant vector according to claim 7, wherein the polynucleotide is a siRNA or a guide RNA for use in CRISPR/Cas9 system.

28. The host cell according to claim 8, wherein the host cell is an embryonic stem cell, an induced pluripotent stem cell or a hematopoietic stem/progenitor cell.

29. The host cell according to claim 28, wherein the induced pluripotent stem cell is a recombinant BC1 cell or a recombinant Aicas9 cell.

30. The kit according to claim 10, wherein the Notch pathway inhibitor is a tumor necrosis factor-α-converting enzyme inhibitor or a γ-secretase inhibitor.

31. The kit according to claim 10, wherein the induced pluripotent stem cell is a recombinant BC1 cell or a recombinant Aicas9 cell.

32. The kit according to claim 13, wherein the polynucleotide is a siRNA or a guide RNA for use in CRISPR/Cas9 system.

33. The method according to claim 14, wherein the method comprises:
the step of using an effective amount of a composition comprising a host cell and cell culture medium, wherein the host cell comprises a recombinant vector comprising a polynucleotide for completely or partially knocking out NOTCH4 gene, or wherein the host cell has NOTCH4 gene completely or partially knocked out; or
the step of using an effective amount of any one of the following items (1) to (4):
(1) a drug for inhibiting or reducing the expression of NOTCH4 gene;
(2) a drug for inhibiting or blocking the activity of NOTCH4 protein;
(3) a drug for completely or partially knocking out NOTCH4 gene; and
(4) a Notch pathway inhibitor.

34. The method according to claim 33, wherein the Notch pathway inhibitor is a tumor necrosis factor-α-converting enzyme inhibitor or a γ-secretase inhibitor.

35. The method according to claim 15, wherein the method comprises:
the step of using an effective amount of a composition comprising a host cell and cell culture medium, wherein the host cell comprises a recombinant vector comprising a polynucleotide for completely or partially knocking out NOTCH4 gene, or wherein the host cell has NOTCH4 gene completely or partially knocked out; or
the step of using an effective amount of any one of the following items (1) to (4):
(1) a drug for inhibiting or reducing the expression of NOTCH4 gene;
(2) a drug for inhibiting or blocking the activity of NOTCH4 protein;
(3) a drug for completely or partially knocking out NOTCH4 gene; and
(4) a Notch pathway inhibitor.

36. The method according to claim 35, wherein the Notch pathway inhibitor is a tumor necrosis factor-α-converting enzyme inhibitor or a γ-secretase inhibitor.

37. The method according to claim 23, wherein the disease associated with abnormal platelet is thrombocytopenia.

38. The method according to claim 23, wherein the Notch pathway inhibitor is a tumor necrosis factor-α-converting enzyme inhibitor or a γ-secretase inhibitor.

39. The method according to claim 26, wherein the polynucleotide is a siRNA or a guide RNA for use in CRISPR/Cas9 system.
